(19) United States
(12) Patent Application Publication
(10) Pub. No.: US 2026/0124334 A1
(43) Pub. Date: May 7, 2026

(54) HOUSING FOR VOLATILE COMPOSITION DISPENSER

(71) Applicant: The Procter & Gamble Company, Cincinnati, OH (US)

(72) Inventors: Rahul VYAS, Singapore (SG); Desmond NG, Singapore (SG); Kumaresh GOVINDAN RADHAKRISHNAN, Singapore (SG); Mei San Gigi CHO, Singapore (SG); Hyunjoon YOON, Singapore (SG); Walter SORDO, Trento (IT); Lung Yee TANG, Singapore (SG); Danilo ROSSI, Trento (IT)

(21) Appl. No.: 19/353,659

(22) Filed: Oct. 9, 2025

Related U.S. Application Data
(60) Provisional application No. 63/710,111, filed on Oct. 22, 2024.

(51) Int. Cl. A61L 9/12

(52) U.S. Cl. CPC A61L 9/12; A61L 2209/131; A61L 2209/133; A61L 2209/15

(57) ABSTRACT

Provided herein is a housing for a volatile composition dispenser that includes:
a front portion and a back portion, where the back portion comprises at least one airflow opening, and the front and back portions together define an interior space for receiving a volatile composition cartridge;
one or more rupturing protrusions extending from an interior surface of the back portion towards the interior space, the one or more rupturing protrusions for directly rupturing a sealing substrate of a volatile composition cartridge;
one or more supporting protrusions extending from an interior surface of the front portion towards the interior space, the one or more supporting protrusions for supporting a volatile composition cartridge in order to facilitate rupturing of the sealing substrate by the one or more rupturing protrusions; and
a cartridge insertion opening that allows a volatile composition cartridge to be slidably received by the interior space.

### FIELD OF THE INVENTION

The present invention relates to the field of volatile composition dispensers, particularly to a housing for a volatile composition dispenser, and to a volatile composition dispenser comprising the housing and a volatile composition cartridge.

### BACKGROUND OF THE INVENTION

Systems for delivering volatile materials to the atmosphere are well known in the art. Such systems include insect repellents, air fresheners, malodor removal agents, or the like, and function by evaporating a volatile material into a space to deliver a variety of benefits such as air freshening or malodor removal.

Most volatile composition dispensers are configured for one-time use. Typical disposable air freshener dispenser devices are described in PCT Publication No. WO 98/16262 and U.S. Pat. No. 10,561,755 B2, which include an air freshener medium within a container, and a push button actuator which can be manually operated to rupture a foil covering the container for initiating the dispensing of the air freshener into the atmosphere. A problem associated with such device is that after volatile composition is depleted, it is not possible to refill or replenish the volatile composition and reactivate the volatile composition dispenser. As a result, the entire product is disposed of, contributing to the environmental problem of plastics waste.

Furthermore, existing commercially available membrane-based volatile composition dispensers are typically intended for use in a high airflow environment such as a room or car air vent. Such products are not very suitable for low airflow environments such as the interior of a waste bin or cupboard, since they may not provide sufficient volatilization of the volatile composition contained therein. There is a consumer need for membrane-based volatile composition dispensers that are adapted for use in these environments, particularly to treat malodors that tend to accumulate in dirty enclosed spaces.

In addition, some volatile composition dispensers include a rupture mechanism, as disclosed in U.S. Pat. Nos. 10,561,754, 10,561,755 and 10,561,756. Rupture mechanisms significantly reduce the forces required to activate a volatile composition cartridge, facilitating activation by a user. However, the inclusion of these rupture mechanisms presents a risk of accidental premature activation, for example if a volatile composition dispenser or cartridge is dropped during the supply chain.

While products that do not comprise a rupture mechanism are known (e.g. in TWI698262B), such products involve a movable rupturing element that impacts a membrane in an orthogonal direction, thereby transferring force onto a sealing substrate located behind the membrane. However, such products are single-use products where a volatile composition cartridge cannot be replaced within the same housing. Consumers do not want to touch a housing to open and close it in a dirty environment, so an openable version of these products adapted for a bin environment would have low consumer appeal. In addition, the presence of moving parts in a housing presents challenges for reusable products, because moving parts are more likely to break during use. There is a need for membrane-based volatile composition dispensers, and associated housings and cartridges, that allow for a cartridge to easily replaced.

Thus, there is a need for a product that solves some or all of the problems discussed above.

### SUMMARY OF THE INVENTION

The invention, in an example, provides a housing for a volatile composition dispenser, which housing is adapted for easy insertion and removal of a cartridge containing a volatile composition. The housing is configured to activate a cartridge upon insertion, thereby providing a simple and efficient cartridge insertion and removal experience. Furthermore, the cartridge insertion and removal experience allows a consumer to avoid touching the housing, solving the consumer problem of disliking touching dirty environments such as the interior of waste bins. The housing of the invention is adapted for use with cartridges that do not contain a rupture mechanism, and is able to rupture a sealing substrate via slide-in activation. There are no existing products on the market that have solved the problem of activating a cartridge via a slide-in activation without a rupture mechanism.

Therefore, the invention provides the following examples.
1. A housing for a volatile composition dispenser, the housing comprising:
   a front portion and a back portion, where the back portion comprises at least one airflow opening, and the front and back portions together define an interior space for receiving a volatile composition cartridge;
   one or more rupturing protrusions extending from an interior surface of the back portion towards the interior space, the one or more rupturing protrusions for directly rupturing a sealing substrate of a volatile composition cartridge;
   one or more supporting protrusions extending from an interior surface of the front portion towards the interior space, the one or more supporting protrusions for supporting a volatile composition cartridge in order to facilitate rupturing of the sealing substrate by the one or more rupturing protrusions; and
   a cartridge insertion opening that allows a volatile composition cartridge to be slidably received by the interior space.
2. The housing according to clause 1, wherein the one or more rupturing protrusions comprise a rupturing apex for rupturing a sealing substrate of a volatile composition cartridge.
3. The housing according to clause 2, wherein the rupturing apex comprises an arc having an arc radius of less than 0.9 cm,
   optionally wherein the rupturing apex comprises an arc having an arc radius of less than 0.7 cm,
   more optionally wherein the rupturing apex comprises an arc having an arc radius of less than 0.5 cm.
4. The housing according to clause 2, wherein the rupturing apex comprises an arc, and where a chord defined by a sagitta of 1.025 mm has an arc length of from 4 mm to 8.5 mm.
5. The housing according to any one of clauses 2 to 4, wherein the one or more rupturing protrusions comprise a sloped region facing the cartridge insertion opening, where the sloped region is configured such that as a cartridge is inserted into the interior space via the cartridge insertion opening, the sloped region guides the cartridge towards the rupturing apex of the one or more rupturing protrusions.
6. The housing according to any one of the preceding clauses, wherein the one or more rupturing protrusions comprises at least two rupturing protrusions.
7. The housing according to clause 6, wherein the at least two rupturing protrusions are laterally offset from a line bisecting the housing in a longitudinal direction.
8. The housing according to clause 7, wherein the at least two rupturing protrusions are laterally and longitudinally offset from each other.
9. The housing according to any one of the preceding clauses, wherein the one or more supporting protrusions comprise a sloped region facing the cartridge insertion opening, where the sloped region is configured such that as a cartridge is inserted into the interior space via the cartridge insertion opening, the sloped region directs the cartridge towards the one or more rupturing protrusions.
10. The housing according to any one of the preceding clauses, wherein at least one of the one or more rupturing protrusions is closer to the cartridge insertion opening than one of the one or more supporting protrusions.
11. The housing according to any one of the preceding clauses, wherein at least one of (a) and (b) applies:
   (a) a cartridge contacting area of the one or more rupturing protrusions is smaller than a cartridge contacting area of the one or more supporting protrusions; and
   (b) the one or more rupturing protrusions and the one or more supporting protrusions each have an arc defined by a chord at a sagitta of 1.025 mm, and a corresponding arc length of the one or more rupturing protrusions is shorter than a corresponding arc length of the one or more supporting protrusions.
12. The housing according to any one of the preceding clauses, wherein each of the one or more rupturing protrusions and one or more supporting protrusions are arranged in pairs, such that each rupturing protrusion has a corresponding supporting protrusion.
13. The housing according to clause 12, wherein a distal point of each rupturing protrusion is less than 1.5 cm from a distal point of its corresponding supporting protrusion, optionally less than 1 cm from its corresponding supporting protrusion, more optionally less than 0.7 cm from its corresponding supporting protrusion.
14. The housing according to any one of the preceding clauses, further comprising a blocking protrusion extending from an interior surface of the front or back portions, the blocking protrusion for preventing insertion of a volatile composition cartridge in an undesired orientation.
15. The housing according to any one of the preceding clauses, wherein the front portion comprises a window,
   optionally wherein the window is at least partially bordered by one or more guiding protrusions, the guiding protrusions for aligning a volatile composition cartridge as the volatile composition cartridge is inserted into the housing.
16. The housing according to any one of the preceding clauses, wherein:
   the one or more rupturing protrusions each comprise a rupturing apex for rupturing a sealing substrate of a volatile composition cartridge;
   the one or more rupturing protrusions comprise a sloped region facing the cartridge insertion opening, where the sloped region is configured such that as a cartridge is inserted into the interior space via the cartridge insertion opening, the sloped region guides the cartridge towards the rupturing apex of the one or more rupturing protrusions;
   the one or more supporting protrusions comprise a sloped region facing the cartridge insertion opening, where the sloped region is configured such that as a cartridge is inserted into the interior space via the cartridge insertion opening, the sloped region guides an opposing side of the cartridge towards the rupturing apex of the one or more rupturing protrusions; and
   at least one of the one or more rupturing protrusions is closer to the cartridge insertion opening than any of the one or more supporting protrusions.
17. The housing according to clause 16, wherein each of the one or more rupturing protrusions and one or more supporting protrusions are arranged in pairs, such that each rupturing protrusion has a corresponding supporting protrusion.
18. The housing according to any one of the preceding clauses, wherein the housing does not comprise moving parts.
19. A volatile composition dispenser comprising:
   a housing as defined in any one of the previous claims; and
   a volatile composition cartridge, the volatile composition cartridge comprising:
      a reservoir containing a volatile composition;
      a sealing substrate enclosing the reservoir; and
   a membrane enclosing the reservoir, the membrane configured to allow volatilisation of the volatile composition,
   wherein:
   the volatile composition dispenser is configured such that as the volatile composition cartridge is slid into the housing via the cartridge insertion opening, the at least one rupturing protrusion is configured to rupture the sealing substrate without rupturing the membrane.
20. The volatile composition dispenser according to clause 19, wherein the volatile composition cartridge comprises a sloped section on a side of the volatile composition cartridge that is opposing to the membrane,
   where the sloped section is for engaging with the one or more supporting protrusions, such that as the volatile composition cartridge is slid into the housing via the cartridge insertion opening, the sloped section engages with one of the one or more supporting protrusions and directs the volatile composition cartridge in a direction towards the one or more rupturing protrusions, thereby increasing the force exerted by the rupturing protrusion on the sealing substrate as compared to a force exerted when the sloped section is not engaged with one of the one or more supporting protrusions.

In such forms, the invention provides a housing for a volatile composition dispenser, which housing is particularly adapted to allow easy insertion and removal of a volatile composition cartridge without needing to remove the housing from the waste bin or cupboard. This is particularly advantageous when the housing is used in a waste bin, because consumers have a strong preference to minimize physical touch with any product that is contained within the dirty environment of a waste bin, especially a food waste bin.

In addition, the housing is adapted for use with a volatile composition cartridge that does not include a rupture mechanism, and allows activation of a volatile composition cartridge without requiring excessive force.

### BRIEF DESCRIPTION OF THE DRAWINGS

While the specification concludes with the claims particularly pointing out and distinctly claiming the invention, it is believed that the present invention will be better understood from the following description taken in conjunction with the accompanying drawings.

FIG. 1 shows a front perspective view of a housing according to the invention.

FIG. 2 shows a side view of a housing according to the invention.

FIG. 3 shows a top view of a housing according to the invention.

FIG. 4 shows a back perspective view of a housing according to the invention.

FIG. 5 shows a perspective view of a front portion of a housing according to the invention.

FIG. 6 shows a perspective view of a back portion of a housing according to the invention.

FIG. 7 shows four views of a cartridge configured to be placed inside the housing.

FIGS. 8A, 8B and 8C show cross-sections of three alternative cartridges.

FIG. 9 shows the angle of a sloped section of a cartridge configured to be placed inside the housing.

FIG. 10 shows examples of sealing areas that may be used to generate rupturing areas on a cartridge configured to be placed inside the housing.

FIG. 11 shows how the location of rupturing areas affects the ability for a volatile composition to evaporate when the housing is oriented in different directions.

FIG. 12. shows a front perspective view of a volatile composition dispenser according to the invention.

FIG. 13 shows a side view of a volatile composition dispenser according to the invention.

FIG. 14 shows a top view of a volatile composition dispenser according to the invention.

FIG. 15 shows a back perspective view of a volatile composition dispenser according to the invention.

FIG. 16 shows the relationship between a sagitta, chord and arc length of a protrusion.

FIG. 17 shows the ergonomics of touching a cartridge held within a housing according to the invention, when the housing is adhered to a surface.

FIG. 18 is a graph depicting evaporative properties of a volatile composition dispenser.

### DETAILED DESCRIPTION OF THE INVENTION

Various configurations will now be described to provide an overall understanding of the principles of the structure, function, manufacture, and use of the apparatuses and methods disclosed herein. One or more examples of these configurations are illustrated in the accompanying drawings. Those of ordinary skill in the art will understand that the apparatuses and methods specifically described herein and illustrated in the accompanying drawings are non-limiting example configurations and that the scope of the various configurations of the present disclosure is defined solely by the claims. The features illustrated or described in connection with one example configuration may be combined with the features of other example configurations. Such modifications and variations are intended to be included within the scope of the present disclosure.

In one aspect, the invention relates to a housing for a volatile composition dispenser, the housing comprising:
a front portion and a back portion, where the back portion comprises at least one airflow opening, and the front and back portions together define an interior space for receiving a volatile composition cartridge;
one or more rupturing protrusions extending from an interior surface of the back portion towards the interior space, the one or more rupturing protrusions for rupturing a scaling substrate of a volatile composition cartridge;
one or more supporting protrusions extending from an interior surface of the front portion towards the interior space, the one or more supporting protrusions for supporting a volatile composition cartridge in order to facilitate rupturing of the sealing substrate by the one or more rupturing protrusions; and
a cartridge insertion opening that allows a volatile composition cartridge to be slidably received by the interior space.

The housing may have any combination of the additional features defined herein. The disclosure herein of embodiments that are narrower than this aspect should not be taken to limit the present disclosure to be narrower than this aspect of the invention.

As used herein, the x-direction and y-direction together define the plane that corresponds to a front/back view of the housing, and the z-direction refers to the direction that separates the front and back portions of the housing. When a volatile composition cartridge is located within the housing, the membrane may be substantially parallel to the xy-plane.

The housing is described in detail below, and use of the housing is described in the context of inserting a volatile composition cartridge in which a reservoir containing a volatile composition is enclosed by a sealing substrate, which sealing substrate is enclosed by a membrane. Such configurations are described in more detail below, and require the sealing substrate to be ruptured through the membrane, without rupturing the membrane. However, the housing described herein may also be used with volatile composition cartridges in which a sealing substrate encloses the membrane, though such volatile composition cartridges typically provide inferior evaporation rates because the unruptured portions of the sealing substrate will limit the airflow over the membrane.

### Housing

The housing of the invention is typically a reusable housing, such that when a volatile composition cartridge is depleted of volatile composition, the volatile composition cartridge may be replaced and the housing may be reused with a new volatile composition cartridge.

The housing comprises a front portion and a back portion. The front and back portions may be separate parts that are connected together, or they may be two parts of an integrally formed housing. The front and back portions may constitute the front and back halves of the housing, i.e. the front 50% and the back 50% of the housing, by distance. Therefore, when the housing comprises two separate parts that are joined together, these parts need not correspond exactly to the front and back portions.

When the front and back portions are separate parts, they may be connected together by any appropriate means, including but not limited to ultrasonic welding, a snap-fit connection, or an adhesive. It may be preferable for the front and back portions to be connected together by ultrasonic welding or an adhesive, since this will hold the front and back portions together in a rigid manner, allowing for a cartridge to be held within the housing more securely. In contrast, a snap-fit connection may become loose over time, as the connections may be stretched by forces imparted on the housing by a cartridge. When the front and back portions are integrally formed, the housing may be molded as a single component.

The housing may typically be formed from a plastics material, such as a polypropylene, which may further comprise a reinforcing component, such as glass fiber (e.g. the housing may comprise about 90% polypropylene with about 10% glass fiber, by weight). A person skilled in the art will be aware of other suitable plastics that may be used.

The front and back portions together define an interior space for receiving a volatile composition cartridge. Thus, the interior space is able to receive a volatile composition cartridge.

The back portion may comprise an adhering section. The adhering section is a section of the back portion that either comprises an adhesive, or is configured to be attached to an adhesive (e.g. an adhesive strip). For example, the adhering section may have surface properties that are different to the rest of the back portion, and which surface properties provide a stronger bond with an adhesive (e.g. an adhesive strip). The presence of an adhering section is advantageous over freestanding products because, by securing the housing to a surface, a volatile composition cartridge may be removed from the housing without needing to touch the housing. This is especially advantageous when the housing is used in a waste bin (e.g. secured to the lid or wall of a waste bin), which is a dirty environment that consumers prefer not to touch.

Thus, in some configurations the adhering section may comprise a section that has been subjected to a different surface treatment to the rest of the back portion. Purely by way of example, when the back portion is formed from a plastics material (e.g. polypropylene), the adhering section may be subjected to a plasma or a glossy surface treatment, while the reminder of the back portion may have a different surface finish, such as VDI 27. A skilled person will appreciate that other surface finishes or treatments may be used to provide an adhering section that forms a strong adhesive bond with an adhesive (e.g. an adhesive strip). In some configurations, the adhering section may have a surface roughness measured according to ISO 21920-1:2021 of less than 1 μm, such as less than 0.7 μm, such as less than 0.5 μm, e.g. less than 0.3 μm.

The adhering section may have a higher surface energy than the rest of the back portion. In some configurations, the adhering section may have a Total Surface Energy as measured by ASTM D7490-13(2022) of at least 31 mN/m. In some configurations, the adhering section may have a Polar Ratio, as determined using ASTM D7490-13(2022), of less than 5%. Surprisingly, it has been found that adhering sections having these properties provide improved bonding to adhesive strips.

In some configurations, the adhering section may comprise an adhesive, such as an adhesive strip. Any appropriate adhesive may be used. In order to provide a strong adhesive bond with the surface of a waste bin, which is typically formed from a plastics material (e.g. polyethylene or polypropylene) or a metal (e.g. stainless steel), the adhesive strip may have a Total Surface Energy as measured by ASTM D7490-13(2022) of no more than 25 mN/m. The adhesive strip may have a Polar Ratio, as determined using ASTM D7490-13(2022), of less than 5%.

Suitable adhesive strips include those that provide a 90° Peel Adhesion on stainless steel, as measured by ASTM D3330-04(2018) of at least 30 N/cm. Non-limiting examples of adhesive strips that may be used in the invention include 3M® VHB 4941, 3M® VHB 4950, 3M® VHB 4945, 3M® VHB LSE-160WF. Additional examples of adhesive strips that may be used with the invention include 3M® VHB 5962, 3M® VHB LSE-060WF, 3M® VHB LVO-110BF, and Gorilla® Mounting Tape (Tough & Clear). Yet further examples of adhesive strips that may be used with the invention include 3M® 5962, 3M® 9495LE, 3M® 410M, and 3M® command tape.

When the housing comprises an adhesive strip, it is desirable that the adhesive strip may be removed from a product (e.g a waste bin) after use without leaving a residue. Thus, the adhesive strip may have a Normal Tensile Strength as measured by ASTM D897-08(2016) of at least 500 kPa. The adhesive strip may have an Overlap Shear Strength as measured by ASTM D1002-10(2019) of at least 400 kPa.

The adhesive strip may have any appropriate size, such as an area of from about 2 cm² to about 30 cm², such as from about 5 cm² to about 20 cm², e.g. about 7 cm² to about 15 cm². A skilled person will appreciate that smaller or larger adhesive strips may be suitable in some circumstances.

The housing comprises one or more rupturing protrusions extending from an interior surface of the back portion towards the interior space. The one or more rupturing protrusions are for directly rupturing the sealing substrate of a volatile composition cartridge as the volatile composition cartridge is inserted into the housing. In this context, “directly” may be interpreted as meaning that a force imparted by the one or more rupturing protrusions onto the scaling substrate (whether via direct contact, or through another component such as a membrane) is sufficient to rupture the sealing substrate. Thus, the presence of a rupturing protrusion on the housing of the invention avoids the need for a rupture mechanism within a volatile composition cartridge. This is described in more detail herein. As used herein, unless otherwise specified, the terms “rupturing protrusion” and “rupturing protrusions” refer to the one or more rupturing protrusions in general and should not be taken as limiting the present disclosure to requiring any particular number of rupturing protrusions.

Where the interior surface of the back portion comprises multiple protrusions, the one or more rupturing protrusions may be the protrusions having the greatest height/depth, since such protrusions will typically be the ones that contact the (membrane of a) volatile composition cartridge.

The rupturing protrusions also serve to distance the volatile composition cartridge from the airflow openings, allowing an effective airflow into and within the housing. For the avoidance of doubt, the inner surface of the back portion may comprise additional protrusions that are not the one or more rupturing protrusions. The additional protrusions may serve to support or guide a volatile composition cartridge within the housing.

Since the rupturing protrusions will typically impart a force directly on the membrane of a volatile composition cartridge during use, the protrusions should be of an appropriate size and shape that does not rupture, pierce, or otherwise damage the membrane. Thus, the rupturing protrusions may comprise a cartridge-contacting (e.g. membrane-contacting) surface that is substantially flat or rounded.

Nevertheless, the force imparted by the rupturing protrusions onto the sealing substrate must cause a pressure sufficient to rupture the sealing substrate. In addition, a high contact area will cause a high amount of friction as the membrane is deformed over the rupturing protrusions, and so the rupturing protrusions should not have an excessively high cartridge/membrane contacting area.

The deformation of the membrane by the rupturing protrusions provides additional benefits. First, the deformation increases the friction between the cartridge and housing, ensuring that the cartridge is held securely within the housing. Second, the deformation of the membrane towards an interior of the cartridge reduces the volume of the interior of the cartridge. This results in an increase in the apparent fill level of volatile composition, increasing consumer satisfaction. This advantage applies especially when the sealing substrate is between the membrane and the reservoir, because rupturing of the sealing substrate will allow fluid to pass through the sealing substrate, causing a drop in the fill level of volatile composition upon activation as the volatile composition occupies the space between the sealing substrate and the membrane. This sudden drop in fill level can cause consumers to believe that the volatile composition is disappearing, leading to dissatisfaction. By compressing the membrane, and reducing the interior volume of the cartridge, this drop in fill level can be mitigated, and consumer satisfaction increased.

The rupturing protrusions may have any appropriate size and shape, such as a cubic, cuboidal, cylindrical, conical or polygonal shape that may have straight or curved edges and faces. The rupturing protrusions may have any appropriate size, but may typically extend a maximum distance from the back portion of from about 0.5 cm to about 3 cm (e.g. about 1 to about 2 cm). Typically, the rupturing protrusions may have a non-flat (e.g. rounded) cartridge contacting surface, such as a rupturing apex, which may typically be a rounded apex. The presence of a rupturing apex helps to provide a sufficient pressure to the cartridge to rupture a sealing substrate, as compared to a flat (e.g. plateau-shaped) rupturing protrusion. The presence of a rounded apex, rather than a pointed/sharp apex, reduces the risk of tearing a membrane as a cartridge is slid over the rupturing protrusion.

The rupturing protrusions are typically arranged in a substantially orthogonal direction, relative to the cartridge insertion opening. Thus, the rupturing apex of a rupturing protrusion will typically point in a direction that is substantially orthogonal to the cartridge insertion opening. “Substantially orthogonal” may mean that the rupturing protrusions and cartridge insertion opening arc offset by an angle of from 70° to 90°, from 75° to 90°, from 80° to 90°, from 85° to 90°, or about 90°. In some configurations, substantially orthogonal may refer to an angle of from 80° to 90°. In some configurations, substantially orthogonal may refer to an angle of from 85° to 90°. Thus, a volatile composition cartridge that is slid into the housing through the cartridge insertion opening will slide over the rupturing apex of the rupturing protrusions, rather than abut against the rupturing protrusions.

The above dimensions are suitable for a household waste bin, though a skilled person will appreciate that larger or smaller protrusions may be used, and that the size of the rupturing protrusions may depend on the overall dimensions of the housing.

In some configurations, the rupturing apex has an arc radius of less than 0.9 cm, such as less than 0.7 cm, such as less than 0.5 cm. In this context, when a rupturing apex has an arc radius of less than X, at least 80% (preferably at least 90%, at least 95%, or at least 97%) of a cross-sectional area of the distal section of the rupturing apex (e.g. the part of the rupturing apex defined by a sagitta of 1.025 mm) will fit inside a semicircle having radius X, where the tip of the cross-section of the rupturing apex is overlayed with the tip of the semicircle. For the avoidance of doubt, the rupturing apex does not need to have an apex with a curvature that corresponds to a circle. In such cases, it is sufficient that the required proportion of the distal section of the rupturing apex fits inside a semicircle having the defined radius.

In some configurations, the rupturing apex may comprise an arc. A chord defined by a sagitta of 1.025 mm may define a corresponding arc length of from about 4 mm to about 8.5 mm. In some configurations, a chord defined by a sagitta of 1.025 mm may define a corresponding arc length of from about 4.5 mm to about 8 mm. In some configurations, a chord defined by a sagitta of 1.025 mm may define a corresponding arc length of from about 5 mm to about 7 mm, e.g. about 5.5 mm. For the avoidance of doubt, the term “arc” as used herein does not necessarily refer to a circular arc, and the arc may comprise portions having different curvatures (i.e. different radii) and may also comprise one or more straight portions. The term “arc length” as used herein refers to the perimeter of a region of said arc defined by a chord. A skilled person will understand that, for a given sagitta, a shorter arc length corresponds to a sharper apex and lower contact area with a membrane sliding over the rupturing apex, while a higher arc length corresponds to a less sharp apex and higher contact area with the membrane.

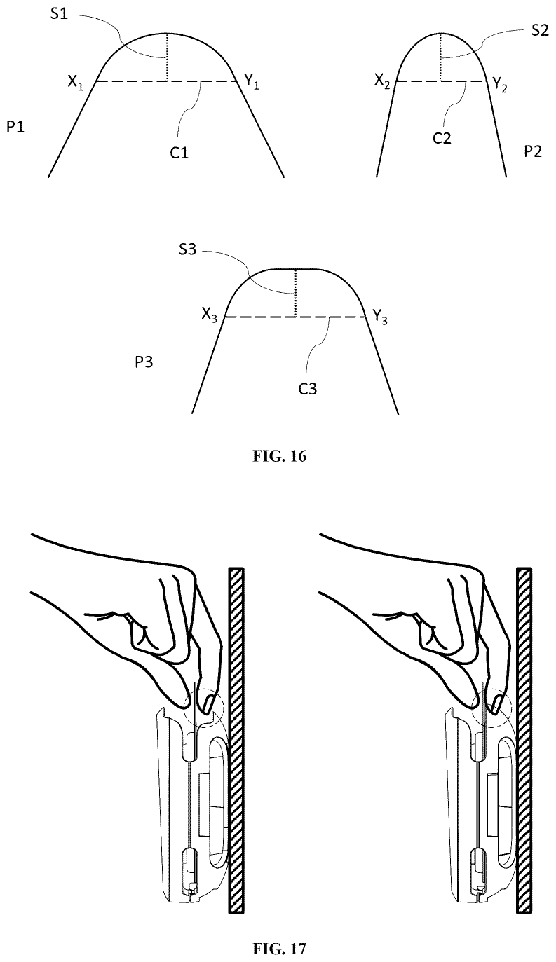

The relationship between an arc length, sagitta and chord is shown in FIG. 16. FIG. 16 shows three protrusions, P1, P2, P3. Each protrusion has a chord C1, C2, C3 respectively defined by sagitta S1, S2, S3. All of sagitta S1, S2, S3 have the same length, but the chords they define result in different arc lengths. Chords C1 and C2 define arcs with curved apexes, with cord C1 defining an arc X₁Y₁, which has a greater arc length than arc X₂ Y₂ defined by chord C2. Chord C3 defines an arc X₃Y₃, which has a flat section at its apex and curved sections either side of the flat section. While the chords C1 and C3 are the same length, arc X₃Y₃ has a longer arc length than arc X₁Y₁. Thus, for any given force, protrusion P3 will impart the lowest pressure onto a cartridge, while protrusion P2 will impart the greatest pressure onto a cartridge.

When the rupturing apex has a larger arc length/radius, it will have a high area in contact with the membrane. This will increase the friction as the membrane passes over the rupturing protrusion, and will also impart a lower pressure onto the rupturable substrate of a volatile composition cartridge, which may require a greater deformation/higher force before rupturing the sealing substrate. This will lead to an undesirably high insertion force. In addition, when the rupturing apex has too small an arc length/radius, it will become sharp and may pierce, tear or otherwise damage the membrane.

The presence of a rupturing apex with a sufficiently small arc length/arc radius ensures that the rupturing protrusions will be able to rupture the sealing substrate of a volatile composition cartridge as it is slid over the rupturing protrusions, without requiring excessive force. Prior art housings, for example that described in WO2010120960A1 and WO2010120961A2, are configured for use with a cartridge that comprises a rupture mechanism. The presence of a rupture mechanism means the housings described therein do not need to impart a force onto a sealing substrate, so they are designed with very large arc lengths/arc radii. The arc length of the housing defined in these prior art publications at a sagitta of 1.025 mm is greater than 9 mm. When a rupturing protrusion has an arc length of this size, the force required to insert a volatile composition cartridge and rupture a sealing substrate without a rupture mechanism, is too high.

The housing comprises an insertion opening for inserting a volatile composition cartridge. For example, the insertion opening may allow a volatile composition cartridge to be slidably received by the interior space. This advantageously allows a volatile composition cartridge to be inserted and removed from the housing without touching the housing. This is desirable because consumers do not like touching a housing that is placed inside a waste bin, because it is perceived as dirty.

In order to facilitate insertion and removal of a volatile composition cartridge by sliding it in and out via the insertion opening, the housing may comprise features that guide the volatile composition cartridge to a desired location during insertion. Thus, housing may comprise various features to guide a volatile composition cartridge into a desired location for rupturing the sealing substrate and so that it may be held securely within the housing. Such features may include, but are not limited to, a sloped region of the rupturing protrusions, one or more supporting protrusions extending from an interior surface of the front portion towards the interior space, a guiding wall, and a blocking protrusion, each of which are discussed in more detail hereinbelow. These features advantageously increase the case and speed of insertion. This is especially important to consumers when the housing is intended to be placed in a waste bin, which is considered to be a dirty and smelly environment and consumers do not want to touch any parts for a long time, and want to minimise the time for which a bin lid is open.

The rupturing protrusions may comprise a sloped region facing the cartridge insertion opening, where the sloped region is configured such that as a cartridge is inserted into the interior space via the cartridge insertion opening, the sloped region guides the cartridge towards the rupturing apex of the one or more rupturing protrusions. In other words, as a part of a cartridge impacts the sloped region during insertion, it will slide up the sloped region and over the rupturing apex, thereby guiding the cartridge into a desired position.

The one or more rupturing protrusions may comprise only a single rupturing protrusion. In this case, the rupturing protrusion may be located at a midline (i.e. a line bisecting the housing in a longitudinal direction) of the housing, or may be laterally offset from the midline. If a single rupturing protrusion is offset from the midline, the housing may preferably comprise another protrusion offset from the midline in the opposite direction, to balance a cartridge within the housing and prevent it from twisting.

The one or more rupturing protrusions may comprise at least two rupturing protrusions. The presence of two rupturing protrusions may have several advantages. First, the two rupturing protrusions may be laterally offset from a midline of the housing, thereby providing two rupturing areas on a sealing substrate of a volatile composition cartridge. This may better balance a cartridge during insertion into the housing, especially when the two rupturing protrusions are equidistant from the midline.

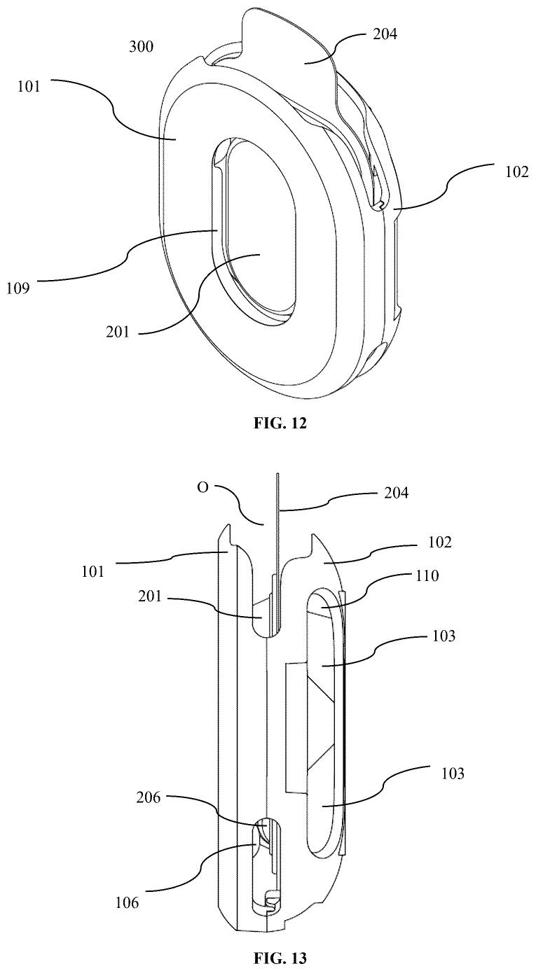

Advantageously, the housing may be configured to rupture a scaling substrate at two opposing corners of the sealing substrate. As shown in FIG. 11, this ensures that, for multiple orientations of a cartridge, there will be a ruptured area of the sealing substrate at a gravitationally lowest position. This is advantageous because it ensures that a liquid present in the volatile composition cartridge is able to flow through the ruptured area of the sealing substrate for all common orientations of the sealing substrate. In order to achieve this, the housing may have two rupturing protrusions that are both laterally and longitudinally offset from each other.

While it is possible to achieve the same orientation benefit by rupturing the sealing substrate along a portion of two opposing edges of the sealing substrate (e.g. with rupturing protrusions that are laterally offset from each other and proximal to the cartridge insertion opening), this will drastically increase the force required to insert the cartridge, and resulting in a product that is difficult to use.

The housing comprises one or more supporting protrusions extending from an interior surface of the front portion towards the interior space, the one or more supporting protrusions for supporting a volatile composition cartridge in order to facilitate rupturing of the sealing substrate by the one or more rupturing protrusions. The supporting protrusions serve to support the volatile composition cartridge and help guide it to a desired position so that the rupturing protrusions may rupture the sealing substrate. As used herein, unless otherwise specified, the terms “supporting protrusion” and “supporting protrusions” refer to the one or more supporting protrusions in general and should not be taken as limiting the present disclosure to requiring any particular number of supporting protrusions.

The one or more supporting protrusions may have any appropriate size and shape, such as a cubic, cuboidal, cylindrical, conical or polygonal shape that may have straight or curved edges and faces. The supporting protrusions may have any appropriate size, but may typically extend a maximum distance from the front portion of from about 0.3 cm to about 3 cm (e.g. about 0.5 to about 1.5 cm). Typically, the supporting protrusions may have a rounded or flat profile at a distal region in the z-direction. This reduces the pressure imparted onto a cartridge by the supporting protrusions, helping direct the cartridge onto the rupturing protrusions without damaging the cartridge. Thus, the supporting protrusions may have a rounded apex or plateau at a distal region in the z-direction. The presence of a rounded profile, rather than a profile with pointed/sharp edges, reduces the risk of damaging the cartridge as it is slid over the supporting protrusion.

The supporting protrusions may help to compress a cartridge onto the rupturing protrusions, i.e. the cartridge may be compressed between the rupturing protrusions and the supporting protrusions as the cartridge is slid into the housing. This compression may lead to rupturing of the scaling substrate by the rupturing protrusions. In some configurations, the one or more supporting protrusions may comprise a sloped region facing the cartridge insertion opening, where the sloped region is configured such that as a cartridge is inserted into the interior space via the cartridge insertion opening, and passes over the sloped region, the sloped region directs the cartridge (e.g. an opposing side of the cartridge, such as a membrane of a cartridge) towards the one or more rupturing protrusions (e.g. towards the rupturing apex of the one or more rupturing protrusions).

In some configurations, at least one of the one or more rupturing protrusions is closer to the cartridge insertion opening than one of the one or more supporting protrusions. In this context, “closer” may be understood as meaning that a distal part of the rupturing protrusion in the z-direction (e.g. an apex) is closer to the cartridge insertion opening than a distal part (e.g. apex or plateau) of the supporting protrusion. Thus, as a volatile composition cartridge is inserted into the housing, it will first encounter the aforesaid rupturing protrusion. As it passes over this rupturing protrusion, the rupturing protrusion will contact and slide over the membrane of the cartridge. At the same time, the rupturing protrusion may cause a deformation of the membrane. As the cartridge subsequently encounters one of the supporting protrusions, the supporting protrusion will direct the cartridge towards the rupturing protrusion that is in contact with the membrane, thereby compressing the cartridge onto the rupturing protrusion. This deforms the membrane, and causes the rupturing protrusion to impart a force onto the sealing substrate (through the membrane), rupturing the scaling substrate. Thus, having one of the supporting protrusions further from the opening than one of the rupturing protrusions helps ensure that the volatile composition cartridge is effectively ruptured by the rupturing protrusion.

The rupturing and supporting protrusions are typically configured to secure the volatile composition cartridge therebetween, such that when fully inserted, the volatile composition cartridge is held securely within the housing. Typically, the housing is configured to secure the volatile composition cartridge without adhesion or attachment, such that the cartridge may be held securely but may be removed simply by pulling the cartridge out of the housing.

The rupturing and supporting protrusions may be arranged in pairs, such that each rupturing protrusion has a corresponding supporting protrusion. In some configurations, a pair may comprise a rupturing protrusion and a supporting protrusion, where the distal point of the rupturing protrusion (i.e. the furthest point of the rupturing protrusion from the back portion, in the z-direction) and a distal point of the supporting protrusion (i.e. the furthest point of the supporting protrusion from the front portion, in the z-direction) are within 1.5 cm from each other in 3D space, such as within 1 cm from each other, or within 0.7 cm from each other in 3D space. Ensuring that the distal points of the rupturing and supporting protrusions are close to each other ensures that the forces imparted into the scaling substrate are maximised. In contrast, if the rupturing and supporting protrusions are far away from each other, then as a result of the lever effect, the cartridge will be more able to flex in response to the forces imparted by the rupturing and supporting protrusions, which reduces the effective force imparted onto the sealing substrate.

Where the rupturing protrusions comprise a rupturing apex, and are arranged in pairs with the supporting protrusions, each rupturing apex may be offset from its corresponding supporting protrusion by less than 0.5 cm, such as less than 0.3 cm, such as less than 0.2 cm, in the z-direction,

The rupturing protrusions may have a smaller cartridge contacting area than the supporting protrusions. As used herein, a cartridge contacting area is an area at a distal region of the protrusion in question that could reasonably be expected to come into contact with a cartridge, as the cartridge slides over the protrusion. Thus, the rupturing protrusions may be configured to impart a higher pressure onto the cartridge than the supporting protrusions. This helps to ensure that the supporting protrusions simply support the cartridge and do not substantially deform it, while the rupturing protrusions rupture the sealing substrate. In some configurations, the rupturing protrusions may comprise a rupturing apex, while the supporting protrusions may comprise a plateau shape. In some configurations, the one or more rupturing protrusions and the one or more supporting protrusions each have an arc defined by a chord at a sagitta of 1.025 mm (e.g. from an apex or plateau), and a corresponding arc length of the one or more rupturing protrusions is shorter than a corresponding arc length of the one or more supporting protrusions. As mentioned above, the term “arc” as used herein does not necessarily refer to a circular arc, and the arc may comprise portions having different curvatures (i.e. different radii) and may also comprise one or more straight portions. The term “arc length” as used herein refers to the perimeter of a region of said arc defined by a chord.

In order to facilitate insertion of a volatile composition cartridge, the insertion opening may have a width that allows easy access to a volatile composition cartridge by a consumer's fingers. Thus, the insertion opening may have a width of at least 10 mm, such as at least 13 mm. In some configurations, the insertion opening may have a width of from 13 mm to 25 mm, for example from 15 mm to 20 mm. Nevertheless, a skilled person will appreciate that other sized insertion openings, especially wider insertion openings, may be appropriate in certain circumstances.

In some configurations, the width of the insertion opening may be at least 50%, such as at least 60%, or at least 70% of the depth of the housing.

The housing is configured such that when it is attached to a surface, a user will have enough space to easily insert and remove a volatile composition cartridge from the housing. For example, a distance between a part of the cartridge gripped by a user and the surface should be large enough for a user's finger to fit between the cartridge and the surface. As shown in FIG. 17, a user may use a pinch grip utilising their thumb and forefinger when inserting or removing a cartridge from the housing, especially when a volatile composition cartridge comprises a gripping tab as disclosed herein. When a housing of the invention is attached to a surface, a user's hand approaching the housing with a pinch grip will typically involve a thumb approaching at an angle (e.g. about 30-60°), with their forefinger approaching at an angle that is closer to parallel to the surface. Given the different angles of approach for a thumb and forefinger, in some configurations, a distance between an intersection of the gripping tab with the insertion opening; and the front portion, may be greater than a distance between an intersection of the gripping tab with the insertion opening; and the back portion. In some configurations, the gripping tab may intersect the insertion opening closer to the back portion than the front portion. This advantageously allows a user's thumb to partially enter the housing when gripping a gripping tab, enabling the use of a cartridge having a smaller and more compact gripping tab that does not extend as far outside the housing.

In some configurations, the front and/or back portions of the housing may comprise a cut-out region adjacent to the insertion opening, the cut-out region for allowing access to the gripping tab. The cut-out region may serve to increase the distance between the edges of the front and back portions of the housing, and the gripping tab.

The at least one airflow opening may comprise any appropriate number of airflow openings, such as two or more airflow openings. In some configurations, the at least one airflow opening may comprise two airflow openings. In some such configurations, the adhering section (when present) may be located between the two airflow openings.

The airflow openings may be located at least partially on a side portion of the housing, such that air flowing sideways relative to the housing may enter the airflow openings. Therefore, in some configurations, at least one of the at least one airflow openings is not parallel to the adhering section. The airflow openings may be oriented at an angle relative to the adhering section, such that the airflow openings extend in a direction towards the front portion of the housing. In some configurations, the adhering section occupies an xy-plane, and the at least one airflow opening extends in the z-direction towards the front portion of the housing. The airflow openings may extend from a region adjacent or near to the adhering section, towards the front portion of the housing. The housing may be configured to hold a volatile composition cartridge such that a membrane of the volatile composition cartridge is facing the at least one airflow opening. When the adhering section is adhered to a movable surface (e.g. a waste bin lid or a cupboard door), turbulent air caused by movement of the surface (e.g. opening of the lid or door) will flow along the surface and enter the airflow openings. The air will then pass over the membrane of a volatile composition cartridge located within the housing, enabling effective volatilization of the volatile composition.

Providing at least one of the at least one airflow openings at an angle relative to the adhering section (e.g. on an angled part of the back portion) may also result in a more ergonomic shape for a user to grip the housing when seeking to remove it from a surface. When the airflow openings are located at an angled part of the back portion, a user's fingers may easily reach around the side of the housing and grip it, without being obstructed by the surface.

Therefore, in some configurations, the at least one airflow opening may be present on a section of the back portion that is angled relative to the adhering section.

In some configurations, the at least one airflow opening may have an elongate shape. For example, the at least one airflow opening may have an aspect ratio of at least 2, such as at least 3, or at least 4. Typically, the at least one airflow opening may have an aspect ratio of less than 15, such as less than 10, or less than 7.

As will be appreciated by a person skilled in the art, the at least one airflow opening should have a total area that is sufficient to provide an airflow over the volatile composition cartridge, which airflow is sufficient for evaporation or volatilization of the volatile composition contained therein. Therefore, in some configurations, a total area A_{F} of the at least one airflow opening is at least 7 cm², such as from 7 cm² to 30 cm², for example from 8 cm² to 25 cm², or from 9 cm² to 22 cm². The size of the at least one airflow opening may be determined using commercially available computer-aided design (CAD) software, such as the area measurement tool available in SOLIDWORKS® 2021.

As mentioned herein, the back portion may comprise the at least one airflow opening and an adhering section. In order to maximise the adhesive strength, and maximise airflow through the housing, it is advantageous for the at least one airflow opening and the adhering to have the maximum area possible. Thus, in some configurations, the sum of:
the area of a projection of the at least one airflow opening onto a plane of the adhering section; and
the area of the adhering section,
   is at least 70% (e.g. at least 80%) of the area of a projection of the back portion onto a plane of the adhering section.

In order to ensure that the adhesive strength and airflow are both at an acceptable level, it is advantageous for a ratio of:
the area of a projection of the at least one airflow opening onto a plane of the adhering section; to
the area of the adhering section,
   to be from 2:1 to 1:2, such as 1.5:1 to 1:1.5. In some configurations, it may be advantageous for the area of the airflow openings to be larger than the area of the adhering section, such that the aforementioned ratio is from 2:1 to 1.05:1, such as 1.5:1 to 1.05:1. The use of a smaller adhering section may be offset by the use of a strong adhesive, which allows for the area of the at least one airflow opening to be maximized. In such configurations, the adhering section may comprise an adhesive strip, and the above ratio may apply equally to the area of the adhesive strip (i.e. the area of the adhering section may be substituted by the area of the adhesive strip).

The housing (e.g. a front portion of the housing) may comprise a window. The window advantageously allows the cartridge to be visible from outside the housing, and this may allow for a fill level of volatile composition to be easily determined without opening the housing. The window may be a cut-out portion of the housing (e.g. of the front portion), or the window may be a part of the housing (e.g. of the front portion) that is made from a transparent material. In some configurations, the window may be configured to receive a part of the cartridge, such as the reservoir (or a portion thereof). This helps to securely hold the cartridge in place within the interior space, without requiring any adhesive or other adhering means. In some such configurations, the window may be a cut-out portion of the front portion, which window is configured to receive a part of the cartridge (e.g. the reservoir).

The window may have any appropriate shape. In order to hold the cartridge securely and prevent rotation of the cartridge, the window may have a substantially oval or pill shape. The window may have any appropriate size. For example, the window may have a maximum dimension in the xy-plane of from about 2 cm to about 10 cm, such as 3.5 cm to about 6 cm. The window may have an oval or pill shape and an aspect ratio of from about 1.2 to about 3, such as about 1.5 to about 2.5. The window may comprise rounded corners to assist insertion of a reservoir part of a volatile composition cartridge into the window during insertion of the volatile composition cartridge into the housing.

The housing may comprise one or more guiding protrusions extending from the front portion towards the interior space. The guiding protrusions may serve to guide or align a cartridge as it is inserted into the housing. The guiding protrusions may be configured to support the cartridge within the housing without adhesion. For example, a part of the cartridge may rest on guiding protrusions when the cartridge is held within the housing. This allows a cartridge to be supported and securely held within the housing, but quickly and easily removed by a user because the cartridge is not adhered to the housing. The presence of guiding protrusions increases the case of insertion of a volatile composition cartridge, and ensures that a volatile composition cartridge is naturally aligned to the correct position during insertion. This increases user satisfaction, especially when the housing is placed in a dirty environment such as a bin, where consumers prefer to minimise their exposure time. The guiding protrusions typically enable a user to insert a volatile composition cartridge in only a few seconds, without needing to try multiple times to find a correct alignment.

In configurations where the housing (e.g. a front portion) comprises a window (e.g. a window that is configured to receive a part of the cartridge, such as the reservoir), then at least a part of a perimeter of the window may be bordered by one or more of the guiding protrusions extending towards the interior space. The one or more guiding protrusions may protrude in a substantially orthogonal direction from the window towards the interior space. The one or more guiding protrusions may be configured to guide the reservoir of the cartridge as the cartridge is inserted into the housing, such as through an insertion opening as described herein. The one or more guiding protrusions may be configured to support the cartridge within the housing without adhesion. For example, a part of the cartridge may rest on the one or more guiding protrusions when the cartridge is held within the housing. This allows a cartridge to be supported and securely held within the housing, but quickly and easily removed by a user because the cartridge is not adhered to the housing.

The one or more guiding protrusions may have any appropriate shape, such as a shape that allows the one or more guiding protrusions to support a cartridge within the housing without adhesion. For example, the one or more guiding protrusions may comprise a single solid (e.g. uninterrupted) wall, or may alternatively comprise multiple protrusions (e.g. multiple interrupted sections of wall). A person skilled in the art will appreciate that any of these configurations may be utilized to support or guide a cartridge within the housing.

When the housing comprises one or more guiding protrusions, the one or more guiding protrusions may be configured to contact a part of the cartridge. For example, a part (e.g. a side part) of the one or more guiding protrusions may be configured to contact the reservoir. In addition, a part (e.g. a distal part) of the one or more guiding protrusions may be configured to contact another area of the cartridge, such as a peripheral seal area as described herein.

The housing may also comprise one or more blocking protrusions, which ensure that a volatile composition cartridge may only be inserted in one way. For example, the interior surface of the back portion may comprise a blocking protrusion, which may have a height that is lower than the maximum height of the one or more rupturing protrusions. The one or more blocking protrusions may prevent insertion of the cartridge in an incorrect configuration. For example, the one or more blocking protrusions may ensure that the cartridge can only be inserted with the reservoir facing the front portion, and not with the reservoir facing the back portion.

In some configurations, the housing does not comprise any moving parts. For example, in such configurations the housing does not comprise hinged parts or sliding parts. For the avoidance of doubt, the volatile composition cartridge as described herein is not considered a moving part.

### Volatile Composition Dispenser

The combination of a volatile composition cartridge and a housing may be referred to as a volatile composition dispenser. Thus, placing a volatile composition cartridge into a housing may typically form a volatile composition dispenser, which may be used to release the volatile composition over time.

Therefore, the invention provides a volatile composition dispenser comprising:
a housing as defined in any one of the previous claims; and
a volatile composition cartridge, the volatile composition cartridge comprising:
   a reservoir containing a volatile composition;
   a sealing substrate enclosing the reservoir; and
   a membrane enclosing the reservoir, the membrane configured to allow volatilisation of the volatile composition,
      wherein:
the volatile composition dispenser is configured such that as the volatile composition cartridge is slid into the housing via the cartridge insertion opening, the at least one rupturing protrusion is configured to rupture the sealing substrate without rupturing the membrane.

In this way, the volatile composition cartridge can be used to dispense at least one volatile composition and/or other solution or composition, such as a perfume, a fragrance, and/or an insecticide, for example, to a surrounding area or atmosphere. The volatile composition can comprise a single chemical or a single material that is capable of entering the vapor phase under atmospheric conditions or, more commonly, the volatile composition can comprise a mixture of chemicals and/or materials that are capable of entering the vapor phase under atmospheric conditions.

The housing of the volatile composition dispenser may have any feature that is discussed above in relation to the housing.

As mentioned above, the housing of the volatile composition dispenser comprises an insertion opening that allows the volatile composition cartridge to be slidably received by the interior space.

Typically, the volatile composition dispenser is configured such that the membrane of the volatile composition cartridge faces the back portion of the housing, and contacts the one or more rupturing protrusions. Thus, air entering the at least one airflow opening will flow across the membrane.

The volatile composition dispenser may be intended to be used within an interior space, such as an interior space of a waste bin or cupboard, although the present invention is not limited to such use and those of skill in the art will understand that the volatile composition dispenser can be configured for use in any appropriate environment, and can be configured to dispense any suitable solution, chemical, material, and/or composition.

The volatile composition cartridge present within the volatile composition dispenser may be configured to dispense a volatile composition in a continuous manner without requiring any energy input, i.e. the cartridge (and corresponding housing) may be non-energized. “Non-energized” can mean that the apparatus is passive and does not require to be powered by a source of external energy. The cartridge and any associated housing does not need to be powered by a source of heat, gas, or electrical current, and the volatile composition is generally not delivered by aerosol means (e.g. the cartridge may not include components under an elevated pressure).

Thus, the volatile composition dispenser of the invention is able to passively and continuously release a volatile composition to a surrounding environment, without requiring any energy source or active actuation step (e.g. pressing of an aerosol actuator).

The continuous emission of the at least one volatile composition can be for any suitable length, such as up to 20 days, 30 days, 40 days, 60 days, 90 days, shorter or longer periods, or any period between 10 to 90 days, for example. Of course, composition having greater or lesser volatility may be provided in the cartridge to increase or decrease its useful life. Also, the cartridge's useful life may be dependent on the conditions (i.e., temperature, pressure, moisture content, airflow etc.) in which it operates.

### Volatile Composition Cartridge

The volatile composition dispenser of the invention comprises a volatile composition cartridge. For the sake of brevity, the volatile composition cartridge may be referred to herein as the “cartridge”.

The cartridge is typically a single-use disposable cartridge that contains a volatile composition for release to a surrounding environment, and once a cartridge is depleted of volatile composition it may be disposed of. The cartridge is for placing into the housing of the invention, such that once the cartridge is depleted of volatile composition it may be removed from the housing and replaced by a new cartridge. The use of single-use cartridges with a reusable housing reduces the amount of material contributed to landfill as compared to products that are entirely single-use (i.e. where the housing is single-use), and also uses a lower volume of material (e.g. plastic) during the manufacturing process.

The cartridge comprises a reservoir containing a volatile composition, and a sealing substrate enclosing the reservoir to prevent evaporation or volatilization of the volatile composition before use. The cartridge also comprises a membrane enclosing the reservoir, and typically also enclosing the sealing substrate. The sealing substrate is configured to be ruptured before use.

The reservoir (or a portion thereof) contains the volatile composition and is enclosed by both the membrane and the sealing substrate.

The sealing substrate comprises one or more rupturing areas, where the one or more rupturing areas have increased tautness as compared to a remaining portion of the sealing substrate. The rupturing areas thus represent areas of the sealing substrate that are ruptured more easily than the remaining portion (i.e. areas other than the rupturing areas) of the sealing substrate. For example:
the sealing substrate substantially occupies an xy-plane;
the one or more rupturing areas are configured to rupture when deformed to a distance A in a z-direction;
said remaining portion of the sealing substrate is configured to rupture when deformed to a distance B in the z-direction, where B>A.

The membrane is spaced from the sealing substrate by a distance C, and where the membrane is configured not to rupture when deformed to a distance A+C in the z-direction. Typically, the distance C is less than 2.5 mm, such as less than 2 mm, such as less than 1.5 mm, e.g. from about 1.5 mm to 0.5 mm. This ensures that the membrane does not need to deform too much before the sealing substrate is ruptured, thereby reducing the risk of damage to the membrane.

The sealing substrate may comprise a sealing area in which the sealing substrate is sealed to the reservoir. For example, the sealing substrate may be sealed to a peripheral portion of the reservoir. The area of the sealing substrate that is adjacent to the sealing area will typically have increased tautness, and therefore in some configurations the one or more rupturing areas are each located no more than 1.5 cm from the sealing area, such as no more than 1 cm from the scaling area, such as no more than 0.8 cm from the sealing area, such as no more than 0.7 cm from the scaling area.

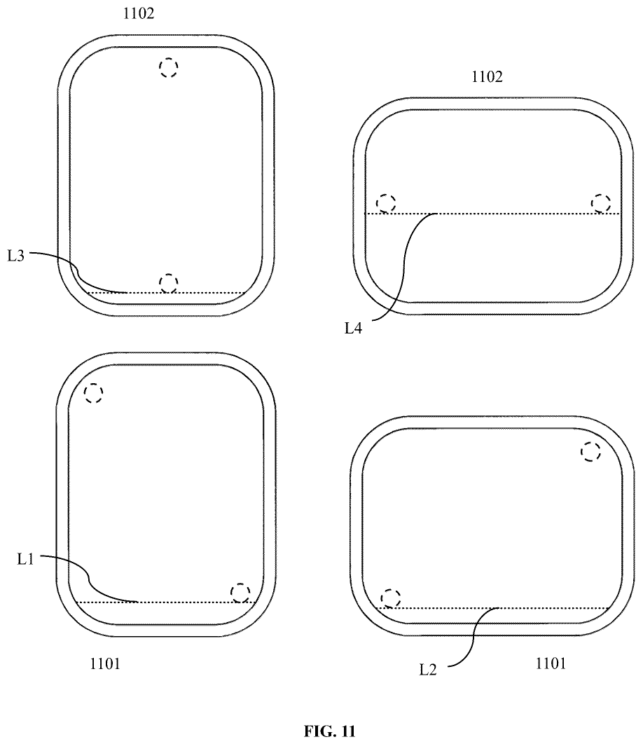

The sealing area typically comprises a perimeter of the sealing substrate. The sealing area may comprise one or more peninsula or island areas protruding into the sealing substrate. The presence of a peninsula or island area will provide a sealing area that surrounds more of a rupturing area, increasing the tautness and facilitating rupturing of the sealing substrate at a lower deformation distance. This is demonstrated in FIG. 10, which is described in more detail hereinbelow.

While the tautness of the sealing substrate may be increased by providing scaling areas, it may also be increased by providing a component in contact with the sealing substrate, which component presses gently onto the sealing substrate, increasing tautness. Thus, in some configurations, the reservoir may comprise one or more reservoir protrusions that contact the sealing substrate, thereby increasing the tautness of the sealing substrate.

In order to ensure sufficient tautness of the one or more rupturing areas, in some configurations the one or more rupturing areas are each surrounded to a cumulative angle of at least 90° within a distance of 1.5 cm, by one or both of the sealing area and an area of the reservoir contacting the reservoir protrusions, such as to a cumulative angle of at least 135°, such as to a cumulative angle of at least 180°. In some configurations, the one or more rupturing areas are each surrounded to a cumulative angle of at least 90° within a distance of 1 cm, by one or both of the sealing area and an area of the reservoir contacting the reservoir protrusions, such as to a cumulative angle of at least 135°, such as to a cumulative angle of at least 180°. In some configurations, the one or more rupturing areas are each surrounded to a cumulative angle of at least 90° within a distance of 0.8 cm (e.g. within a distance of 0.7 cm), by one or both of the sealing area and an area of the reservoir contacting the reservoir protrusions, such as to a cumulative angle of at least 135°, such as to a cumulative angle of at least 180°.

In some configurations, the sealing substrate comprises at least two rupturing areas. This advantageously means that the sealing substrate will be ruptured in two locations upon activation. The presence of two rupturing areas will improve the flow of volatile composition through the scaling substrate, since one rupture point will allow flow of the volatile composition, and another rupture point may allow for pressure to be equalised either side of the sealing substrate.

In some configurations, the scaling substrate comprises at least two rupturing areas that are laterally and longitudinally offset from one another. For example, the sealing substrate may comprise four quadrants (though the quadrants may not be marked on the sealing substrate), the quadrants defined by a latitudinal midline and a longitudinal midline; and the two rupturing areas may be located at opposing quadrants of the sealing substrate. This advantageously means that the cartridge may be oriented in multiple ways and still have a rupture point in the sealing substrate near a gravitationally lowest point of the sealing substrate, allowing for substantially all of the volatile composition to flow through the ruptured scaling substrate. In particular, the presence of two rupturing areas at opposing quadrants may represent rupturing areas in two opposing corners of the sealing substrate (e.g. of a substantially rectangular scaling substrate). Since consumers tend to attach a housing to a surface either vertically or horizontally, but not at an angle therebetween, this results in a rupturing area near a gravitationally lowest point of the scaling substrate, allowing for substantially all of the volatile composition to flow through the ruptured sealing substrate. This is demonstrated in FIG. 11, which is described in more detail hereinbelow.

Where the sealing substrate comprises a sealing area in which the sealing substrate is scaled to the reservoir, said two of the at least two rupturing areas may each be located no more than 1.5 cm from the sealing area, optionally no more than 1 cm from the scaling area, more optionally no more than 0.8 cm from the sealing area. The cartridge described herein advantageously allows a spent or finished cartridge to be removed from the housing without a user needing to touch the membrane, thereby avoiding contact between a user's hands and organic components of the volatile composition throughout the entire cartridge life cycle. This may be achieved by providing a gripping tab on the cartridge, such that a user may hold the gripping tab when inserting and removing the cartridge from the housing. Thus, the cartridge may be secured solely by friction, so that the cartridge can be removed from the housing simply by pulling the gripping tab.

In some configurations, the volatile composition cartridge may comprise a gripping tab.

In some configurations, when the volatile composition cartridge is located within the interior space, the gripping tab may extend beyond the insertion opening to an exterior of the housing. This advantageously improves the case of inserting and removing the volatile composition cartridge from the volatile composition dispenser.

In some configurations, the gripping tab may intersect the insertion opening closer to the back portion than the front portion. This provides a larger gap between the gripping tab and the front portion of the housing, allowing more room for a user's thumb to approach the gripping tab, as shown in FIG. 17.

The gripping tab may have any appropriate size, such that it may be gripped between a user's thumb and forefinger. Thus, the gripping tab may have an area of at least 1 cm². In some configurations, the gripping tab may have an area of at least 1.5 cm², at least 2 cm², at least 2.5 cm², or at least 3 cm². In some configurations, the gripping tab may have an area of from 1.5 cm² to 10 cm², such as from 2 cm² to 9 cm², such as from 2.5 cm² to 8 cm², such as from 3 cm² to 7 cm². The end points of any of these ranges may be combined with any other end point from any other range.

The gripping tab may extend beyond an evaporative edge of the membrane by a distance of at least 0.6 cm, which enables it to be effectively gripped by a user, such as between a user's thumb and forefinger. In this context, “evaporative edge of the membrane” is the edge of the part of the membrane from which volatile composition is able to evaporate. The evaporative edge of the membrane may correspond to the edge of the membrane, but a skilled person will appreciate that a membrane may be configured in such a way that the volatile composition is only able to evaporate from a part of the membrane. Thus, the gripping tab constitutes a part of the cartridge that a user may grip when inserting the cartridge into, and removing the cartridge from, a housing. When touching the gripping tab, a user will not touch an area of the membrane that is wetted with volatile material. In some configurations, the gripping tab may extend beyond an evaporative edge of the membrane by a distance of at least 0.8 cm. In some configurations, the gripping tab may extend beyond an evaporative edge of the membrane by a distance of at least 1 cm. In some configurations, the gripping tab may extend beyond an evaporative edge of the membrane by a distance of at least 1.3 cm. In order to ensure that the cartridge is not overly large, the gripping tab may extend beyond an evaporative edge of the membrane by a distance of from 0.8 cm to 5 cm, such as 1 cm to 3 cm, such as 1.3 cm to 2.3 cm.

Typically, the gripping tab may have an area that is not more than 30% of the evaporative surface area of the membrane, for example an area that is not more than 20% of the evaporative surface area of the membrane. As used herein, “evaporative surface area” is to be understood as the area of the membrane from which a volatile composition may evaporate (e.g. when the membrane is wetted with volatile composition). The evaporative surface area of the membrane may correspond to the area of the membrane.

Limiting the area of the gripping tab relative to the membrane allows the area of the membrane to be maximized for any given size of cartridge. By maximizing the area of the membrane, and providing a small but effective gripping tab, the cartridge may provide excellent evaporative performance, whilst also maintaining case of use by not being overly large. In some configurations, the gripping tab may have an area of not more than 10 cm².

The area of the gripping tab may be calculated based on the area that extends beyond an evaporative edge of the membrane at the gripping end. In some configurations, the membrane may have a straight edge, and the gripping tab may extend beyond the straight edge and form an external gripping tab. In other configurations, the membrane may have a cut-out region that is replaced with the gripping tab, such that the gripping tab does not extend beyond the outermost regions of the membrane, and the area of the gripping tab may correspond to the area of the cut-out region.

In some configurations, the gripping tab may be substantially coplanar with the membrane. Since the membrane may not be entirely planar, this may be determined relative to a part of the reservoir to which the membrane is sealed, e.g. the gripping tab may be substantially coplanar to a part of the reservoir to which the membrane is scaled. When a housing comprises an insertion opening for sliding a cartridge into the housing, the cartridge will typically be inserted with the membrane parallel to the direction of insertion. Thus, when the gripping tab is substantially coplanar with the membrane, it is aligned substantially parallel to the direction of insertion, enabling easy gripping when a user is inserting and removing the cartridge from a housing. In addition, this configuration may be advantageously straightforward to manufacture because the gripping tab may be made from a piece of material to which the membrane is attached, but where the gripping tab corresponds to a part of said material that extends beyond an evaporative edge of the membrane. Furthermore, since the cartridge may typically be slid into a housing through a slot-like opening, elements of the housing (such as protrusions described herein) may impart forces onto the membrane, such as frictional forces and other forces. When the gripping tab is substantially coplanar with the membrane, it is substantially coplanar with the point on the cartridge that these forces act on, reducing torque applied to the cartridge by a user pushing via the gripping tab. “Substantially coplanar” may mean that the parts in question are either parallel or offset by an angle of less than 5° (e.g. less than 3°), and either coplanar or offset by an average distance of less than 3 mm (e.g. less than 2 mm).

In some configurations, the gripping tab may be substantially orthogonal to the membrane and aligned in a longitudinal direction relative to the cartridge. Thus, if the membrane occupies an xy-plane, and the reservoir extends in the z-direction, the gripping tab may occupy a yz-plane. This configuration may allow a user to grip the gripping tab without needing to reach behind it, thereby avoiding being obstructed by a surface to which the housing is attached.

In some configurations, the gripping tab may be formed from the same material as the reservoir. In some configurations, the gripping tab and reservoir may be integrally formed. The gripping tab and reservoir may be formed from a plastics material, such as polyethylene terephthalate (PET), which plastics material may be thermoformed.

Advantageously, the gripping tab may be formed from a rigid material such as a plastics material (e.g. a thermoformed plastics material), since this will allow for the cartridge to be held via the gripping tab during insertion into a housing. In contrast, a flexible gripping tab will flex when pushed, and so will not be effective for pushing a cartridge into a housing.

In order to ensure that the gripping tab has an appropriate rigidity to enable the cartridge to be pushed into a housing, the gripping tab may be a sheet of plastics material (e.g. PET), and may have a thickness of at least 250 microns, such as from 250 microns to 2000 microns, e.g. from 300 microns to 1000 microns.

The gripping tab may comprise a textured surface, which may help increase a user's grip on the gripping tab. The gripping tab may comprise one or more holes, such as a hole large enough for a user's finger to pass through and hold the gripping tab.

The gripping tab allows a user to hold the cartridge easily without touching the membrane, and hence, the cartridge advantageously allows a spent or finished cartridge to be inserted into, and removed from, the housing without a user needing to touch the membrane.

The cartridge may be secured in the housing solely by friction, so that the cartridge can be removed from the housing simply by pulling the gripping tab.

In order to facilitate rupturing of the sealing substrate during insertion of the cartridge into a housing, the cartridge may comprise a sloped section, the sloped section having increasing depth with increasing distance from the insertion end. Typically, the sloped section may be distinct from a main body of the reservoir, though the sloped section may be adjoined to the reservoir. Thus, a maximum height of the sloped section, measured perpendicular to the membrane, is typically no greater than a maximum height of the reservoir. The sloped section is typically configured to interact with a protrusion on a corresponding housing (e.g. a supporting protrusion as discussed herein), such that as the sloped section engages with the (supporting) protrusion, further insertion of the cartridge causes the membrane side of the cartridge to move away from the (supporting) protrusion, towards an opposing side of the housing. When the opposing side comprises rupturing protrusions, the presence of the sloped section of the cartridge facilitates the rupturing of the sealing substrate by the rupturing protrusions, by increasing a force imparted onto the scaling substrate by the rupturing protrusions.

While the slope of the sloped section may not be uniform, the sloped section typically defines a maximum angle of less than 25° (such as less than) 20° relative to the membrane. Since the membrane may not be entirely planar, for this purpose the angle may be taken relative to a part of the reservoir to which the membrane is sealed. This ensures that the insertion force is not overly high. A higher angle for the sloped section will drastically increase the insertion force as the sloped section interacts with the housing.

In some configurations, the sloped section terminates at an apex. This advantageously provides a gradual increase in resistance to insertion as a sloped section engages with a protrusion on a housing, as the sloped section slides over the protrusion. When the sloped section is overcome, i.e. when the apex passes the protrusion, there will be a sudden decrease in resistance to insertion, and an accompanying click sound. This provides a clear signal to a user that the cartridge has been inserted properly.

The cartridge may comprise an abutment, which may be formed from a part of the reservoir, though is typically distinct from a main body of the reservoir. The abutment may be configured to abut with a corresponding structure on a housing (e.g. a supporting protrusion), to prevent over-insertion of the cartridge.

As explained herein, the cartridge of the invention is configured such that the sealing substrate may be ruptured directly by one or more rupturing protrusions on a housing. In order to facilitate this rupturing, the sealing substrate is preferably close to the membrane, and the space between the sealing substrate and membrane preferably does not comprise any solid elements. For example, in some configurations the cartridge does not comprise a rupture mechanism located between the sealing substrate and the membrane. In this context, a rupture mechanism may refer to a rupture mechanism of the sort disclosed in U.S. Pat. Nos. 10,561,754, 10,561,755 and 10,561,756. In some configurations, the cartridge does not comprise a sealing substrate-supporting element located between the sealing substrate and the membrane. In this context, a sealing substrate-supporting element refers to an element located between the membrane and scaling substrate, which element is configured to support the sealing substrate and facilitate rupturing of the sealing substrate.

The reservoir, membrane, and sealing substrate are discussed in turn below.

### Reservoir

The reservoir (or a portion thereof) contains the volatile composition, and has an opening that is enclosed by the membrane and sealing substrate.

The reservoir of the cartridge may typically be formed from a plastics material, which may advantageously be transparent to allow an easy view of a fill level of volatile composition within the reservoir. An example of a suitable material is polyethylene terephthalate (PET).

The reservoir may be configured for interfacing with the housing described herein. In some configurations the term “interfacing” may be understood as meaning that at least a portion of the reservoir is configured to be received by a window of a housing, so that the cartridge is held securely within the housing when the housing is closed. The reservoir (or a portion thereof) may therefore have a shape that is configured to correspond with a window of a housing, so the reservoir (or a portion thereof) may be received and fit snugly within the window. Advantageously, when the reservoir (or a portion thereof) of a cartridge is received by a window of a housing, a user is provided with a clear signal that the cartridge has been inserted correctly.

In configurations in which the window is intended to receive (part of) a reservoir of a cartridge, it may be advantageous for the reservoir to be formed from a transparent material, so that the fill level of volatile composition within the reservoir is visible from outside the housing, such as through the window.

The cartridge disclosed herein is typically a single-use cartridge for placing into a housing. Thus, the cartridge typically does not comprise a housing of its own. Therefore, the reservoir of the cartridge may be an outermost layer of the cartridge. In this context, “outermost” is to be understood as meaning that the cartridge does not include a substantial component outside the reservoir. For the avoidance of doubt, this does not exclude the presence of the membrane and sealing substrate enclosing an opening of the reservoir. In some configurations, the reservoir may nevertheless include a label or wrapping around the reservoir, which is intended to convey information to a user. However, the reservoir may be transparent as discussed herein, and in such cases the reservoir may typically not be covered by an additional label or wrapping so as to not obscure the reservoir.

The reservoir may have any appropriate shape. In order to hold the cartridge securely and prevent rotation of the cartridge, the reservoir may have a profile that corresponds to a shape of a window in a housing with which the cartridge is configured to be used. Thus, the reservoir (or a portion thereof) may have a substantially oval or pill shape. The combination of a window and reservoir (or a portion thereof) both having an oval or pill shape ensures that the cartridge is held securely within the window and cannot rotate within the window. In addition, the absence of corners/vertices allow for the cartridge to be placed and removed from the window more easily than a polygonal shape which requires specific alignment. When an oval or pill shaped cartridge is placed into an oval or pill shaped window, the curved edges of the cartridge and window may naturally align the cartridge during insertion, improving ease of use and user experience.

The reservoir may have any appropriate size. For example, the reservoir may have a maximum dimension in the xy-plane of from about 2 cm to about 10 cm, such as about 3.5 cm to about 6 cm. In order for the reservoir to be securely held within a window of a housing, without the possibility of rotation, the reservoir may comprise a portion having an oval or pill shape, which portion is configured to be received by the window. These shapes enable the tapered geometry discussed above, without sharp edges. An oval or pill shaped portion may have an aspect ratio of from about 1.2 to about 3, such as about 1.5 to about 2.5. The dimensions may be measured at a maximum distance away from the membrane, in a plane that is parallel to the plane of the membrane. As discussed herein, the reservoir may comprise rounded corners/edges to assist insertion of a reservoir part of a volatile composition cartridge into a window of a housing during insertion of the volatile composition cartridge into the housing.

In some configurations, the reservoir may comprise a gripping tab-end portion at an end of the reservoir closest to the gripping tab; an opposing-end portion at an end of the reservoir furthest from the gripping tab; and a middle portion therebetween. Each of these portions, i.e. each of the gripping tab-end portion, the opposing-end portion, and the middle portion, have a depth perpendicular to the membrane.

In some configurations, the depth of the opposing-end portion decreases with increasing distance from the gripping tab. This forms a tapered opposing-end portion that is less deep at the insertion end, improving the case of inserting the cartridge into an insertion opening of a housing. Thus, the cartridge may have a generally increasing depth from the insertion end at least until the middle portion of the reservoir.

In some configurations, the depth of the middle portion may decrease with increasing distance from the gripping tab. This forms a tapered middle portion that is less deep at the insertion end, improving the case of inserting the cartridge into an insertion opening of a housing. In other configurations, the depth of the middle portion may be consistent throughout its length.

The gripping tab-end portion may have a depth that increases with distance from the gripping tab. The reservoir may comprise a transition region between the middle portion and the gripping tab-end portion, and the transition region may have a rounded surface profile, rather than forming a point. This enables a smooth transition between the middle portion and the gripping tab-end portion. The rounded surface profile may assist with inserting and removing the cartridge from a housing, especially when the housing has a protrusion that is intended to contact the reservoir and secure the reservoir in place. For example, the housing may comprise a protrusion that contacts the reservoir as the cartridge is slid into the housing. Friction between the protrusion and the reservoir will exert a resistance during insertion, until the transition region passes the protrusion, after which the cartridge may “click” into place and be secured within the housing. The presence of the rounded transition region facilitates the passing of the reservoir past the protrusion, enabling easy insertion and removal of the cartridge without the cartridge becoming stuck against the protrusion during removal. In contrast, a non-rounded transition region may result in a sharp edge or point, requiring greater force to insert and remove, and may also be more prone to damage/deformation.

In some configurations, a maximum depth of the reservoir may be greater than a depth of the cartridge at the insertion end.

In some configurations, the reservoir has a length L measured along a midpoint of the reservoir in a direction from the insertion end to the gripping end, and a maximum depth of the reservoir is located at the middle portion or the transition region. In some configurations, a maximum depth of the reservoir may be located at the middle portion. In some configurations, a maximum depth of the reservoir may be located at the transition region. In some configurations, a maximum depth of the reservoir is located at least 0.5 L (e.g. at least 0.7 L) from an end of the reservoir closest to the insertion end.

As mentioned above, in some configurations the reservoir may have a maximum dimension in the xy-plane of from about 2 cm to about 10 cm, such as about 3.5 cm to about 6 cm. In some configurations the reservoir may have a maximum depth in the z-direction of from about 5 mm to about 20 mm, such as from about 6 mm to about 15 mm, where the depth is measured perpendicular to the membrane. In some specific configurations, the reservoir may have a maximum depth of from about 6 mm to about 9 mm, such as about 6.5 mm to about 8 mm. In other specific configurations, the reservoir may have a maximum depth of from about 10 mm to about 15 mm, such as about 11.5 mm to about 13.5 mm. In some configurations, the reservoir may have a maximum depth in the z-direction that is from 7% to 35% of the maximum dimension of the reservoir in the xy-plane.

The above depth ranges are advantageous because they result in a sturdy reservoir that displays improved resistance to buckling and denting, whilst still being sufficiently large to contain enough volatile composition to provide a sustained release and counteract malodor in a waste bin for up to 8 weeks. In contrast, reservoirs that have much greater depth may not have the structural integrity to resist buckling when products are dropped during the supply chain or in retail stores. Reservoirs that have a lesser depth may not be able to contain sufficient volatile composition to provide a sustained counteracting of malodor for 8 weeks.

In some configurations, the transition region may be located at least 0.5 L (e.g. at least 0.7 L) from an end of the reservoir closest to the insertion end.

In some configurations, the reservoir may comprise all of the following:
a gripping tab-end portion at an end of the reservoir closest to the gripping tab;
an opposing-end portion at an end of the reservoir furthest from the gripping tab;
a middle portion between the gripping tab-end portion and the opposing-end portion;
a transition region between the gripping tab-end portion and the middle portion,
   wherein:
each of the gripping tab-end portion, the opposing-end portion, and the middle portion, have a depth perpendicular to the membrane, the depth of the opposing-end portion decreases with increasing distance from the gripping tab, the depth of the gripping tab-end portion increases with increasing distance from the gripping tab;
a maximum depth of the reservoir is located at the middle portion or the transition region; and
the transition region is located at least 0.5 L (e.g. at least 0.7 L) from an end of the reservoir closest to the insertion end.

The above-described geometry of the cartridge, and particularly the reservoir, facilitates insertion of the cartridge into a housing, particularly insertion into a slot-type opening. Similar principles may apply to the width of the reservoir and/or cartridge.

In some configurations, a maximum width of the reservoir may be greater than a width of the reservoir closest to the insertion end. By providing a narrower part of the reservoir closest to the insertion end, it may be easier to initially insert the insertion end of the cartridge into an opening of a housing. Therefore, in some configurations, a width of the reservoir at the opposing-end portion may decrease with increasing proximity to the insertion end. In addition, or alternatively, the opposing-end portion may comprise a narrowing width taper towards the insertion end.

In addition to the variable width and depth of the reservoir discussed above, the reservoir may comprise a trapezoid-like taper in both length and width as the reservoir extends away from the membrane. This taper improves case of insertion of the reservoir into a window of a housing. Typically, the taper at the longitudinal ends of the reservoir may have a lower angle relative to the membrane than that at the transverse ends of the reservoir. The transverse ends of the reservoir may have a small taper such that the side walls are close to 90° to the membrane (e.g. greater than 70°). This helps to reduce the overall volume of the reservoir for any given height and width, meaning that the reservoir appears to have a greater fill level for a given volume, increasing consumer satisfaction. The longitudinal ends of the reservoir may have a greater taper, e.g. the longitudinal side walls may be at an angle of from 30-70° to the membrane, allowing for a wedge-like shape at the ends of the reservoir, facilitating insertion and removal of the cartridge. The respective angles may be measured along a midpoint of the reservoir.

### Membrane

The volatile composition is in liquid form and is configured to evaporate through a membrane. Accordingly, the cartridge comprises a membrane, which for the sake of brevity may be referred to herein as “the membrane”. The membrane may enclose the reservoir (or a portion thereof) such that volatile composition is unable to escape from the cartridge without passing through the membrane. The membrane may prevent the passage of liquid, such that the volatile composition is only able to escape the cartridge by evaporating through, or from, the membrane.

The membrane may be microporous or monolithic. In some configurations, the membrane may be microporous. Microporous membranes become impregnated with liquid volatile composition, which may evaporate from the membrane. The rate limiting step for microporous membranes is the evaporation of the volatile composition from the membrane. In contrast, monolithic membranes do not become impregnated with liquid volatile composition, but are porous to gas phase volatile composition, such that volatile composition that evaporates inside the reservoir may diffuse through a monolithic membrane and this diffusion is the rate limiting step. Thus, microporous membranes provide advantageously improved perception of volatile composition because when a waste bin lid, or cupboard door, is opened, there will be a sudden increase in airflow over the membrane, which will cause a sudden increase in evaporation of the volatile composition. This effect is not seen with monolithic membranes because the airflow outside the membrane does not increase the rate of evaporation of volatile composition inside the reservoir. Therefore, when the volatile composition comprises a perfume, the use of a microporous membrane will advantageously cause a user to perceive a greater amount of the perfume when opening the bin lid or cupboard door, increasing user satisfaction.

The membrane is vapor permeable and may be capable of wicking liquid, yet prevents free flow of liquid out of the membrane. Any suitable membrane may be used. Purely by way of example, certain properties that may result in advantageous membranes are discussed below. However, the invention is not limited to membranes having the properties below, and any membrane known in the art that allows the volatile composition to evaporate may be used in the invention.

The membrane may have any appropriate volume average pore diameter, such as from 0.01 μm to 0.5 μm, such as from 0.02 μm to 0.3 μm, such as from 0.05 μm to 0.2 μm, more particularly from 0.065 μm to 0.15 μm since this may provide improvements with regard to evaporation rate and controlling leakage or sweating of volatile composition. In certain configurations, the membrane may have a volume average pore diameter of from 0.065 μm to 0.15 μm, from 0.07 to 0.12 μm, from 0.07 to 0.11 μm, or 0.08 to 0.1 μm.

In some configurations, the membrane may have a pore size distribution such that at least 50%, such as at least 60%, such as at least 70%, such as at least 80% or such as at least 90% of the pores of the membrane have a pore diameter of from 0.065 μm to 0.15 μm.

The membrane may comprise (e.g. be formed from) any appropriate material, such as polyethylene, such as ultra-high molecular weight polyethylene (UHMWPE), though other length polyethylene chains may also be used. As used herein, UHMWPE refers to polyethylene having a molecular mass of from about 3.5 million to 7.5 million amu.

The membrane may have a thickness in the z-direction, of about 0.01 mm to about 1 mm, alternatively between about 0.2 mm to about 0.4 mm, from about 0.22 to about 0.37 mm, e.g. from about 0.25 to about 0.35 mm.

The membrane may be formed from a single piece, or single sheet, of material. In other words, the membrane may not be laminated. Thus, the membrane may be formed from a single sheet of polyethylene having a thickness as described above.

Those of ordinary skill in the art will appreciate that the surface area of the membrane can vary depending on the user preferred size of the cartridge. In some configurations, the (evaporative) surface area of the membrane may be about 2 cm² to about 100 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm².

Particularly preferred membranes may have an evaporative surface area of from about 15 cm² to about 40 cm², such as from about 20 cm² to about 35 cm².

The membrane may form substantially all (e.g. at least 80%, at least 85%, at least 90% or at least 95%) of the surface area of a face of the cartridge. Thus, the cartridge may have a front face and a back face, and the membrane may form substantially all (e.g. at least 80%, at least 85%, at least 90% or at least 95%) of the surface area of the front or back face of the cartridge. In some configurations, the membrane may have an area that is at least 80%, at least 85%, at least 90% or at least 95% of a projection of the cartridge onto a plane, where the plane is selected to provide the maximum area. This advantageously allows the membrane to have a maximised evaporative surface area for the size of the cartridge, leading to improved release of volatile composition.

Thus, in some configurations the membrane may have an evaporative surface area of from about 15 cm² to about 40 cm², such as from about 20 cm² to about 35 cm² and form substantially an entire face of the cartridge.

In some configurations, the membrane may have an evaporative surface area of from about 15 cm² to about 40 cm², such as from about 20 cm² to about 35 cm² and the cartridge may have a maximum dimension of less than 11 cm, preferably less than 10 cm. This advantageously means that the cartridge has a compact size whilst retaining a high evaporative surface area.

In such configurations, the membrane has a high size relative to the overall size of the cartridge. It is therefore particularly advantageous for the cartridge to comprise a gripping tab, which enables the cartridge to be removed from a housing without the membrane being touched by a user. Without a gripping tab, it would be very difficult to avoid touching the membrane (which is wetted with volatile composition) when removing the cartridge from a housing. This may be achieved by ensuring that the cartridge is held within the housing without adhesion or attachment, so a user can simply pull the gripping tab, and does not need to disengage or unstick the cartridge from the housing.

The membrane may have any appropriate porosity. For example, the membrane may have a porosity of from 45% to 70%, on a volume basis, such as from 45% to 65%. In certain configurations, the porosity may be from 50 to 70%, such as 55 to 65%.

The membrane may have any appropriate total pore volume, such as from 0.6 to 2 cm³/g. Typically, the total pore volume may be from 0.65 to 1.6 cm³/g, such as 0.7 to 1.5 cm³/g. In certain configurations, the total pore volume may be from 0.8 to 1.4 cm³/g.

The membrane may have any appropriate bulk density, such as from 0.3 to 0.8 g/cm³. Typically, the bulk density may be from 0.35 to 0.75 g/cm³, such as from 0.4 to 0.7 g/cm³. In certain configurations, the bulk density may be from 0.4 to 0.6 g/cm³.

Suitable membranes for the present invention include polyethylene membranes having the properties described herein, available from Microporous, LLC.

The membrane may comprise any suitable filler and plasticizer known in the art. Fillers may include finely divided silica, clays, zeolites, carbonates, charcoals, and mixtures thereof. In one configuration, the membrane may be filled with about 30% to about 80%, by total weight, of silica.

In one aspect of the invention, the membrane may include a dye that is sensitive to the amount of volatile composition it is in contact with to indicate end-of-life. Alternatively, the membrane may change to transparent when in contact with a fragrance or volatile composition to indicate diffusion is occurring. Other means for indicating end-of-life that are known in the art are contemplated for the present invention.

The membranes described herein may advantageously provide a clear visual change when wetted with volatile composition, and when dry (whether before use or at end of life). Such visual changes may be more detectible when the membrane does not comprise a white pigment (e.g. TiO₂). Therefore, the membrane may comprise less than 5 wt. % of a white pigment, such as less than 1 wt. % of a white pigment, less than 0.1 wt. % of a white pigment, or less than 0.01 wt. % of a white pigment. The membrane may be free from a white pigment.

The visual change when the membrane is wetted as compared to dry may be more noticeable when the membrane comprises a coloured dye/pigment or a black dye/pigment. Therefore, the membrane may comprise a coloured or black dye/pigment, such as activated charcoal. Such a coloured or black pigment/dye (e.g. activated charcoal) may be present in any suitable amount, such as from 0.1 to 5 wt. %, e.g. 0.3 to 1 wt. %.

### Scaling Substrate

As mentioned herein, the cartridge comprises a sealing substrate that encloses the reservoir (or a portion thereof), and hence, encloses the volatile composition. This prevents evaporation of the volatile composition for as long as the sealing substrate is intact. The sealing substrate may be ruptured to allow the volatile composition to evaporate. This rupturing may be referred to herein as “activating” or “activation of” the cartridge. The cartridge is configured to be activated before use, i.e. the scaling substrate is configured to be ruptured before use. In this context, “use” refers to enabling the volatile composition to evaporate from the cartridge, i.e. perform its function of dispensing the volatile composition by evaporation. In other words, rupturing the sealing substrate enables evaporation of the at least one liquid volatile composition from the cartridge.

Non-limiting examples of suitable sealing substrates include an impermeable film, foil, or laminate, such as a flexible (e.g. polymeric) film, a flexible (e.g. metal) foil, or a composite material (e.g. a foil/polymeric film laminate). The impermeable film, foil or laminate is provided adhered to the cartridge to prevent evaporation of volatile composition. A particular example of a suitable scaling substrate is aluminium foil.

The sealing substrate may have any appropriate thickness, such as from 10 μm to 1 mm, from 15 μm to 100 μm, from 18 μm to 50 μm, or from 20 μm to 35 μm.

There are two possible configurations for the order of the sealing substrate and the membrane.

In some configurations, the sealing substrate may be positioned between the membrane and the volatile composition, initially preventing the volatile composition from contacting the membrane. In such configurations, the sealing substrate is configured to be ruptured before use. The rupturing may be caused by a force imparted onto the sealing substrate by an external component acting through the membrane. In such cases, the sealing substrate may be located adjacent to the membrane, such that the membrane requires only a very small degree of deformation until it makes contact with the sealing substrate.

Thus, the housing comprises rupturing protrusions as described herein that are configured to activate the cartridge by rupturing the sealing substrate through the membrane. The activation may occur when a cartridge is slid into the housing, where the rupturing protrusions press against the membrane as it is slid into the housing, and hence apply a force to the sealing substrate.

Once the sealing substrate is ruptured, the volatile composition may pass through the ruptured sealing substrate and come into contact with the membrane. The volatile composition may then impregnate the membrane, from which it may evaporate.

In other configurations, the membrane may be positioned between the volatile composition and the sealing substrate, such that the volatile composition is in contact with the membrane but cannot evaporate and leave the cartridge because the membrane is enclosed by the scaling substrate. In such configurations, the sealing substrate will be ruptured by direct contact with the rupturing protrusions. However, depending on the location of the rupturing protrusions, this may result in only small holes in the sealing substrate. The unruptured portions of the sealing substrate will limit the airflow over the membrane, thereby limiting the rate at which the volatile composition may evaporate from the cartridge. Nevertheless, a skilled person will appreciate that a cartridge having this configuration may be used with a housing as described herein that comprises multiple rupturing protrusions near the cartridge insertion opening, to create multiple tears along the sealing substrate along its length during insertion of the cartridge. This may provide sufficient airflow for evaporation of the volatile composition from the membrane through the tears/holes in the scaling substrate. In addition, a further disadvantage of this configuration is that the sealing substrate, if an outermost layer of the cartridge, will be vulnerable to damage or rupture during a supply chain, leading to a risk of premature activation.

The cartridge may comprise a peripheral seal area, in which a peripheral portion of at least two of the reservoir, the membrane, and the sealing substrate may be sealed together. The peripheral seal area may comprise only a peripheral portion of the reservoir and membrane. In such configurations, the cartridge may also comprise an inner peripheral seal area in which an inner peripheral portion of the reservoir is sealed to the sealing substrate. This may be achieved by providing the reservoir with an intermediate step at an inner peripheral portion (e.g. between the peripheral portion and a main body of the reservoir), where the sealing substrate may be sealed to the intermediate step of the reservoir. Either of the peripheral seal area and the inner peripheral seal area described in this paragraph may include the sealing area described above, in which the sealing substrate is sealed to the reservoir.

### Interaction Between Housing and Cartridge

In some configurations, a peak force required to insert the cartridge into the housing may be from about 5 to about 40 Newtons, such as about 10 to about 25 Newtons.

In some configurations, a peak force required to remove the cartridge from the housing may be from about 3 to about 25 Newtons, such as about 5 to about 20 Newtons.

The peak insertion and removal force may be determined as follows.
1. Secure and rigidly support the housing with the housing in a vertical orientation.
2. Place a cartridge gently in vertical orientation at the opening of the housing.
3. Use a flat test tip on an IMADA digital force gauge (or equivalent) to push the cartridge at a rate of 100 mm/min until cartridge is fully inserted into the housing. Record the peak insertion force.
4. Use a grip tip on an IMADA digital force gauge (or equivalent) to grip the cartridge and pull the cartridge at a rate of 100 mm/min until cartridge is fully extracted from the housing. Record the peak extraction force.
5. Repeat steps 1-4 for five different housings and cartridges and determine the average insertion and extraction forces.

The housing and cartridge may together be configured such that insertion of the cartridge produces an audible click sound when the cartridge is in its intended insertion position. Such a click sound may be achieved by having the reservoir slide over a part of the housing before being received by the window. This may slightly deform the reservoir and/or housing during insertion, and once the reservoir is received by the window, the reservoir and/or housing may revert to their original shape, and produce a click sound.

In some configurations, the cartridge may comprise a sloped section configured to interact with the housing as the cartridge is slid into the housing, such that when the cartridge reaches a predetermined point, the sloped section interacts with a part of the housing (e.g. a corresponding protrusion) to produce an audible sound. Such a sloped section may be located at any appropriate location on the cartridge, such as on the reservoir, or separate to the reservoir.

The housing may also comprise a sloped section that interacts with the sloped region during the insertion process. For example, the housing may comprise a supporting protrusion as described herein, which supporting protrusion has a sloped region facing the cartridge insertion opening. The sloped section of the cartridge, and sloped region of the supporting protrusion each engage with a part of the other of the cartridge and housing (typically the sloped section and sloped region engage with each other). The presence of a sloped section on the cartridge provides a gradual increase in height with distance from the insertion end of the cartridge. The presence of a sloped region on a supporting protrusion on the housing provides a gradual increase in height with distance from the cartridge insertion opening of the housing. These provide a steady increase in resistance to insertion as the cartridge is slid into the housing. However, when the sloped region of the supporting protrusion is fully overcome by the corresponding part of a cartridge (e.g. the sloped section), the resistance will suddenly decrease, providing feedback to the user that the cartridge has reached a desired location. This sudden decrease in resistance will typically be accompanied by a click sound, as the engaging of the cartridge and housing will generally cause a deformation of the cartridge and/or housing (typically the cartridge), and reversion of the deformation will typically cause a click sound.

The sloped section and sloped region discussed above typically have a smooth/gradual increase in height, and not a stepped increase. The sloped region typically forms a straight or curved slope. The sloped section typically terminates at an apex. The sloped region typically terminates at an apex.

Insertion of the cartridge may produce a sound of at least 35 dB, such as at least 40 dB, at least 45 dB, or at least 50 dB.

The evaporation rate of volatile composition will depend on the relative airflow over the membrane, as well as the nature of the volatile composition. Certain properties of the volatile composition dispenser may be important to ensure a sufficient evaporation rate of volatile composition, including a total area of the at least one airflow opening, a distance from the surface on which the volatile composition dispenser is attached to the membrane, and a total evaporative area of the membrane. The first two of these properties affect the airflow over the membrane, while the total evaporative area of the membrane affects how much volatile composition may evaporate for any given airflow.

The distance from the surface on which the volatile composition dispenser is attached, to the membrane, may be approximated as the distance from the adhering section to the membrane. Since the membrane and the adhering section may not be parallel, the distance between the membrane and the adhering section may be measured by determining a mean distance from the adhering section to an orthographic projection of the adhering section onto the membrane.

In order to provide good evaporation rates within low airflow, and high humidity, environments such as a waste bin, the volatile composition dispenser may have any or all of the following properties.
An evaporative surface area of the membrane (A_{E}), in cm², may be at least 9, such as at least 15, such as at least 20.
An area (A_{F}) of the at least one airflow opening, in cm², may be at least 7, such as at least 8, such as at least 9.
An orthographic projection of the adhering section onto the membrane defines an area A2, and a mean distance D2 from the adhering section to the area A2, measured in mm orthogonal to the adhering section, may be at least 5, such as at least 7, such as at least 10.

The interrelation between these three properties will affect an evaporation rate of volatile composition. As will be appreciated by a person skilled in the art, if one property has a low value, this may be offset by increasing one or both of the other values. For example, a low distance D2 may be offset by increasing the areas A_{E} and/or A_{F}. Therefore, in some configurations the product:

$$D 2 \times {A}_{E} \times {A}_{F}$$

may be at least 1100 (such as at least 2000, at least 2500, at least 3000, at least 3500, at least 4000 or at least 4500). In some specific configurations, the product D2×AE×AF may be at least 2000. In some specific configurations, the product D2×AE×AF may be at least 4000. In some specific configurations, the product D2×AE×AF may be at least 4500.
Particular ranges for the properties include the following.
An evaporative surface area of the membrane (A_{E}), in cm², may be from 9 to 40, such as from 15 to 40, such as from 20 to 40.
An area (A_{F}) of the at least one airflow opening, in cm², may be from 7 to 25, such as from 8 to 25, such as from 9 to 22.
An orthographic projection of the adhering section onto the membrane defines an area A2, and a mean distance D2 from the adhering section to the area A2, measured in mm orthogonal to the adhering section, may be from 5 to 30, such as from 7 to 30, such as from 10 to 30.

Since an airflow opening that is too large may cause the volatile composition to evaporate too quickly, in some configurations it may be desirable for the airflow openings to have an area that is appropriately sized for the membrane of the volatile composition cartridge. Therefore, in some configurations, a ratio of the total area (A_{F}) of the at least one airflow opening to the evaporative surface area of the membrane (A_{E}), is from 1:3 to 1:1.

### Volatile Composition

The volatile composition can comprise, but is not limited to, a substance that can function as an air freshener, a deodorant, an odor neutralizing material, an odor blocking material, a malodor counteractant, an odor masking material, an aromatherapy material, an aromachology material, an insecticide, air and/or surface sanitizer, and/or a combination thereof. In other various configurations, the volatile composition can comprise other various materials that can act in their vapor phase to modify, enhance, and/or treat an atmosphere or an area outside of the cartridge.

The term “volatile composition” as used herein, refers to a material that is vaporizable at room temperature and atmospheric pressure without the need of an energy source. The volatile composition may be a composition comprised entirely of a single volatile material. The volatile composition may also be a composition comprised entirely of a volatile material mixture (i.e. the mixture has more than one volatile component). Further, it is not necessary for all of the component materials of the composition to be volatile. Any suitable volatile composition in any amount or form, including a liquid or emulsion, may be used.

Liquid suitable for use herein may, thus, also have non-volatile components, such as carrier materials (e.g., water, solvents, etc). It should also be understood that when the liquid is described herein as being “delivered”, “emitted”, or “released,” this refers to the volatilization of the volatile component thereof, and does not require that the non-volatile components thereof be emitted.

The volatile composition can be in the form of perfume oil. Most conventional fragrance materials are volatile essential oils. The volatile composition can be a volatile organic compound commonly available from perfumery suppliers. Furthermore, the volatile composition can be synthetically or naturally formed materials. Examples include, but are not limited to: oil of bergamot, bitter orange, lemon, mandarin, caraway, cedar leaf, clove leaf, cedar wood, geranium, lavender, orange, origanum, petitgrain, white cedar, patchouli, neroili, rose absolute, and the like. In the case of air freshener or fragrances, the different volatile materials can be similar, related, complementary, or contrasting.

It may be desirable for the volatile composition to be in the form of a liquid at 25° C. As explained herein, the membranes used in the current invention may have advantageously increased visual appearance changes when wetted with volatile composition.

The volatile composition may have a combined vapour pressure of at least 8 Pa at 25° C., such as at least 30 Pa at 25° C.

In addition to, or as part of, the volatile composition, the cartridge may include any known malodor composition to neutralize odors. Suitable malodor compositions include reactive aldehydes and ketones. In particular, the volatile composition may comprise a volatile carbonyl containing compound having a vapor pressure of at least 0.02 torr at 25 degrees Celsius, wherein the volatile carbonyl containing compound is selected from the group consisting of: volatile aldehydes, ketones, and mixtures thereof. In some configurations, the volatile composition may comprise from 7 wt. % to 40 wt. % (e.g. from 10 wt. % to 35 wt. %) of the volatile carbonyl containing compound.

While not wishing to be bound by theory, the continuous delivery of a volatile composition may be a function of various factors including membrane pore size; membrane surface area; the physical properties of a volatile composition, such as molecular weight and saturation vapor pressure (“VP”); and the viscosity and/or surface tension of the volatile composition.

The volatile composition may be formulated such that the composition comprises a volatile material mixture comprising about 10% to about 100%, by total weight, of volatile materials that each having a VP at 25° C. of less than about 0.01 torr; alternatively about 40% to about 100%, by total weight, of volatile materials each having a VP at 25° C. of less than about 0.1 torr; alternatively about 50% to about 100%, by total weight, of volatile materials each having a VP at 25° C. of less than about 0.1 torr; alternatively about 90% to about 100%, by total weight, of volatile materials each having a VP at 25° C. of less than about 0.3 torr. In one embodiment, the volatile material mixture may include 0% to about 15%, by total weight, of volatile materials each having a VP at 25° C. of about 0.004 torr to about 0.035 torr; and 0% to about 25%, by total weight, of volatile materials each having a VP at 25° C. of about 0.1 torr to about 0.325 torr; and about 65% to about 100%, by total weight, of volatile materials each having a VP at 25° C. of about 0.035 torr to about 0.1 torr. One source for obtaining the saturation vapor pressure of a volatile material is EPI Suite™, version 4.0, available from U.S. Environmental Protection Agency.

Two exemplary volatile compositions comprising a volatile material mixture having volatile materials of varying VPs are set forth below in Tables A and B. These compositions are shown by way of illustration and are not intended to be in any way limiting of the invention.

TABLE A

| Wt % | Low VP (torr) | High VP (torr) |
|---|---|---|
| 27.71 | 0.175 | 0.325 |
| 20.78 | 0.0875 | 0.1125 |
| 13.86 | 0.0625 | 0.0875 |
| 8.66 | 0.0375 | 0.0625 |
| 8.66 | 0.0175 | 0.0325 |
| 6.93 | 0.00875 | 0.01125 |
| 6.93 | 0.00625 | 0.00875 |
| 3.18 | 0.00375 | 0.00625 |
| 1.27 | 0.00175 | 0.00325 |
| 0.95 | 0.000875 | 0.001125 |
| 0.64 | 0.000625 | 0.000875 |
| 0.32 | 0.000375 | 0.000625 |
| 0.09 | 0.000175 | 0.000325 |

TABLE B

| Wt % | Low VP (torr) | High VP (torr) |
|---|---|---|
| 33.38 | 0.175 | 0.325 |
| 25.75 | 0.0875 | 0.1126 |
| 19.07 | 0.0625 | 0.0875 |
| 13.86 | 0.0375 | 0.0625 |
| 4.00 | 0.0175 | 0.0325 |
| 1.50 | 0.00875 | 0.01125 |
| 0.50 | 0.00625 | 0.00875 |
| 0.72 | 0.00375 | 0.00625 |
| 0.55 | 0.00175 | 0.00325 |
| 0.27 | 0.000875 | 0.001125 |
| 0.20 | 0.000625 | 0.000875 |
| 0.13 | 0.000375 | 0.000625 |
| 0.07 | 0.000175 | 0.000325 |

The viscosity of a volatile composition may control how and when a volatile composition is delivered to the membrane. For example, less viscous compositions may flow faster than the more viscous volatile compositions. Thus, the membrane may be first wetted with the less viscous materials. To help prevent liquid from seeping through the membrane, volatile compositions may have viscosities less than about 23 cP and surface tension less than about 33 mN/m.

In one embodiment, the volatile composition may have a viscosity of about 1.0 cP to less than about 25 cP, alternatively about 1.0 cP to less than about 23, alternatively about 1.0 cP to less than about 15 cP.

The volatile composition may be designed such that the composition may include a surface tension of about 19 mN/m to less than about 33 mN/m, alternatively about 19 mN/m to less than about 30 mN/m, alternatively about 19 mN/m to less than about 27 mN/m.

Also provided herein is a housing corresponding to that described hereinabove, which housing does not comprise an adhering section. Such housings may comprise a hook, which enables the housing to be hung from an object such as a hook located on a wall.

Also provided herein is the use of a housing as described herein, to secure a cartridge containing a volatile composition in a waste bin or shoe cupboard.

Also provided herein is the use of a volatile composition dispenser as described herein, to release a volatile composition (e.g. to counteract malodor) in a waste bin or shoe cupboard. In some configurations, any of the following properties may apply.
(1) A ratio of an evaporative surface area of the membrane (A_{E}), in cm², to a volume of the waste bin in litres, is at least 0.18, such as at least 0.3, such as at least 0.4.
(2) A ratio of an area (A_{F}) of the at least one airflow opening, in cm², to a volume of the waste bin in litres, is at least 0.14, such as at least 0.16, such as at least 0.18.
(3) An orthographic projection of the adhering section onto the membrane defines an area A2, and a ratio of a mean distance D2 from the adhering section to the area A2, measured in mm orthogonal to the adhering section, to a volume of the waste bin in litres, is at least 0.1, such as at least 0.14, such as at least 0.2.
(4) A ratio of the product D2×A_{E}×A_{F} described herein, to a volume of the waste bin in litres, is at least 22, such as at least 40, such as at least 80, such as at least 90.

In some configurations, volatile composition dispensers for use with large waste bins may preferably have increased evaporative surface areas of the membrane (A_{E}) and airflow opening area (A_{F}), without a large increase in distance D2. This is because a large increase in D2 may make the product overly deep, such that if placed on a waste bin lid it may clash with the sides of the bin, or with refuse located inside the bin. Therefore, in some configurations, a ratio of the square root of the area of the at least one airflow opening (A_{F}) in cm², to the cube root of the waste bin volume in litres, is from 1:2 to 2:1. In some configurations, a ratio of the square root of the evaporative surface area of the membrane (A_{E}) in cm², to the cube root of the waste bin volume in litres, is from 3:1 to 1:1.

Also provided herein is a method of reducing malodor in a waste bin or shoe cupboard, the method comprising:
adhering a housing as described herein to an interior surface of a waste bin or shoe cupboard;
inserting a cartridge containing a volatile composition into the housing; and
allowing the volatile composition to volatilise.

Also provided herein is a method of reducing malodor in a waste bin or shoe cupboard, the method comprising:
adhering a volatile composition dispenser as described herein to an interior surface of a waste bin or shoe cupboard; and
allowing the volatile composition to volatilise.

For the avoidance of doubt, for the purposes of the above methods, where a waste bin is contained within a cupboard or cabinet, the step of adhering a housing or volatile composition dispenser to an interior surface of a waste bin encompasses adhering the housing or volatile composition dispenser to the interior surface of the cupboard or cabinet that contains the waste bin.

Also provided herein is a kit comprising a housing as described herein, and a cleaning wipe. The cleaning wipe may be used to clean a surface to remove grease prior to adhering the housing, which will ensure a stronger adhesive bond is formed. For example, the cleaning wipe may comprise an alcohol such as ethanol or isopropyl alcohol.

Various configurations will now be described to provide an overall understanding of the principles of the structure, function, manufacture, and use of the products disclosed herein. One or more examples of these configurations are illustrated in the accompanying drawings. Those of ordinary skill in the art will understand that the apparatuses and methods specifically described herein and illustrated in the accompanying drawings are non-limiting example configurations and that the scope of the various configurations of the present disclosure are defined solely by the claims. The features illustrated or described in connection with one example configuration may be combined with the features of other example configurations. Such modifications and variations are intended to be included within the scope of the present disclosure.

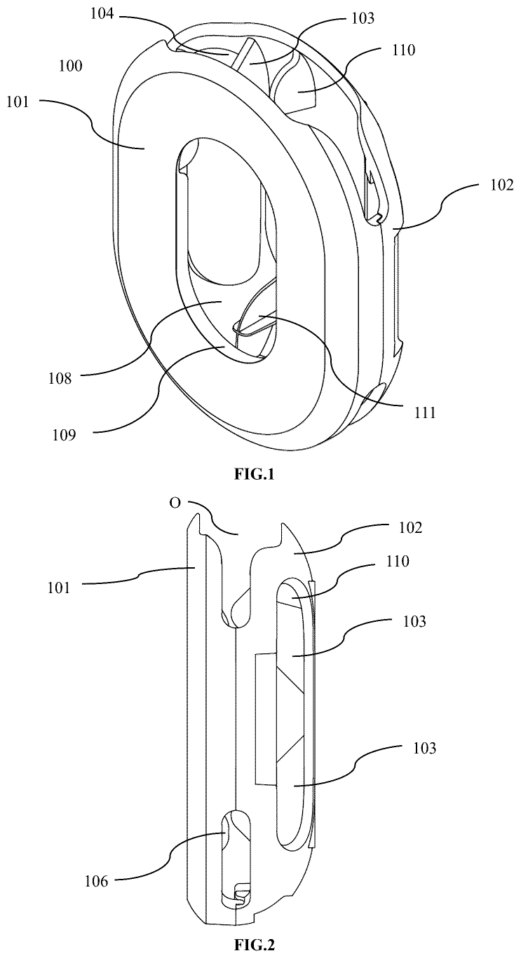

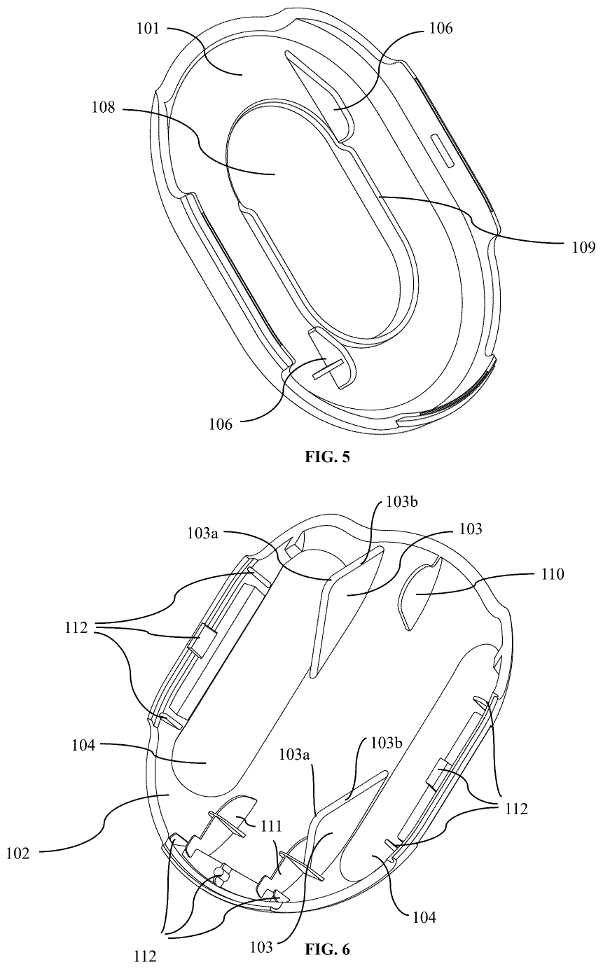

FIGS. 1 to 4 show different views of a housing 100, which includes a front portion 101 and a back portion 102. FIG. 5 shows an interior view of a front portion 101, while FIG. 6 shows an interior view of a back portion 102. The front and back portions together define an interior space, and may be connected by any appropriate means, such as ultrasonic welding, a snap fit connection, or being integrally formed. The back portion 102 comprises an airflow opening 104 (labelled in FIG. 6) and an adhering section 105 (labelled in FIG. 4). The back portion 102 also comprises rupturing protrusions 103, that extend from an interior surface of the back portion 102 towards the interior space. The front portion comprises supporting protrusions 106 extending from an interior surface of the front portion towards the interior space. The housing 100 is configured to secure a volatile composition cartridge comprising a membrane, such that the membrane typically contacts the protrusions 103. The housing 100 comprises a cartridge insertion opening O (FIG. 2) that allows a volatile composition cartridge to be slidably received by the interior space. The rupturing protrusions 103 comprise a rupturing apex 103a, and a sloped region 103b facing the cartridge insertion opening O. The sloped region 103b is configured to guide a cartridge towards the rupturing apex 103a. The front portion 101 comprises a window 108, which is configured to receive a reservoir of a volatile composition cartridge. The window is at least partially bordered by a guiding protrusion 109, which in this embodiment takes the form of a wall. The guiding protrusion 109 guides the reservoir of the cartridge as the cartridge is inserted into the housing. The back portion 102 also comprises a blocking protrusion 110, which serves to prevent insertion of a volatile composition cartridge in an undesired/incorrect orientation. Optional additional protrusions 111 are shown in FIGS. 1 and 6, on which a volatile composition cartridge may rest when fully inserted into the housing.

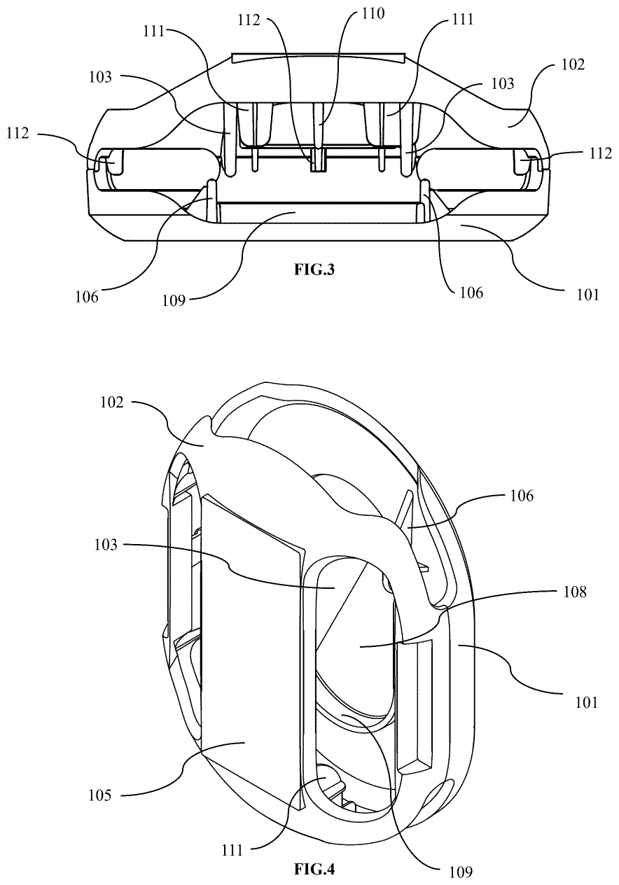

Labelled in FIGS. 3 and 6 are optional parts 112 on the back portion that may be useful in connecting the front 101 and back 102 portions together, for example by ultrasonic welding.

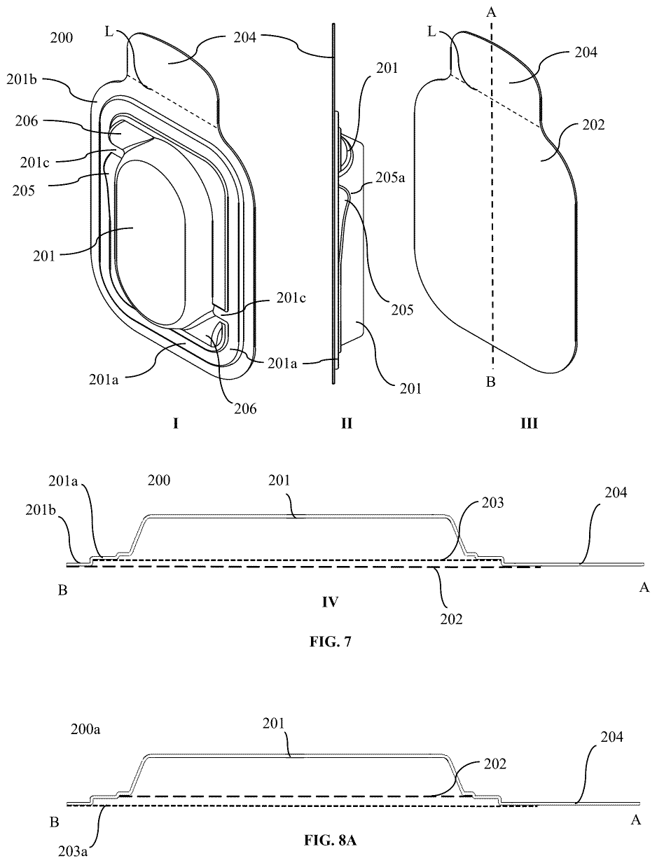

FIG. 7 shows four views of an example of a volatile composition cartridge 200. View I shows a perspective view of the front of the cartridge, View II shows a side view of the cartridge, and View III shows a perspective view of the rear of the cartridge. The cartridge 200 has a gripping end A and an insertion end B. View IV is a cross-section view along the dashed line running from gripping end A to insertion end B in View III. The cartridge 200 comprises a reservoir 201 which contains a volatile composition in liquid form. The reservoir 201 has an opening and is enclosed by a membrane 202, located on the rear of the cartridge. The membrane 202 is shown in View III, and depicted by a dashed line in View IV. The membrane 202 is sealed to a peripheral portion 201b of the reservoir. A sealing substrate 203 (shown by a dashed line in View IV for clarity) is sealed to a shoulder region 201a of the reservoir 201, such that the scaling substrate 203 encloses a portion of the reservoir 201, thereby containing the volatile composition. The sealing substrate 203 is also sealed to a peninsula part 201c of the shoulder region 201a, and the shoulder region 201a and peninsula part 201c together form a sealing area in which the scaling substrate 203 is scaled to the reservoir 201. However, a skilled person will appreciate that peninsula part 201c may simply rest against the sealing substrate without being sealed to the scaling substrate, and it will serve to increase the tautness of the sealing substrate adjacent to peninsula part 201c. The scaling substrate depicted in FIG. 7 is configured to be ruptured before use, for example by rupturing protrusions as described herein and shown in FIG. 6. A gripping tab 204 is located at a gripping end A of the cartridge 200. In the cartridge depicted in FIG. 7, the reservoir 201 and gripping tab 204 are integrally formed. A dashed line L in View III depicts the boundary between the gripping tab 204 and the membrane 202, which are substantially coplanar. Although the membrane 202 is not shown in View I, the dashed line L is replicated in View I to distinguish the gripping tab 204 from the peripheral portion 201b of the reservoir, though the gripping tab 204 and reservoir 201 (including 201a and 201b) may be integrally formed. The cartridge 200 comprises a sloped section 205 having increasing depth with increasing distance from the insertion end B. The sloped section 205 has an apex 205a. The sloped section 205 shown in FIG. 7 is adjoined to the reservoir 201, and a maximum height of the sloped section, measured perpendicular to the membrane, is not greater than a maximum height of the reservoir. The sloped section 205 is configured to interact with a protrusion on a corresponding housing (e.g. a supporting protrusion 106 as shown in FIG. 5), such that as the sloped section 205 engages with the supporting protrusion 106, further insertion of the cartridge causes the membrane side of the cartridge to move away from the supporting protrusion, towards a rupturing protrusion 103 on the opposing side of the housing. The cartridge 200 further comprises raised areas 206, which provides space for a rupturing protrusion to pass through the sealing substrate when it ruptures the sealing substrate. The raised areas 206 are also configured to abut with the supporting protrusions 106 of FIG. 5 to prevent over-insertion of the cartridge 200 into the housing 100. Thus, as the cartridge 200 is inserted into the housing 100, a supporting protrusion 106 interacts with the sloped section 205 to provide a gradually increasing resistance. As the supporting protrusion 106 overcomes the apex 205a of the cartridge 200, the resistance will suddenly decrease, confirming to a consumer that the cartridge 200 is fully inserted into the housing 100. The raised areas 206 will then abut the supporting protrusions 106, preventing further insertion of the cartridge.

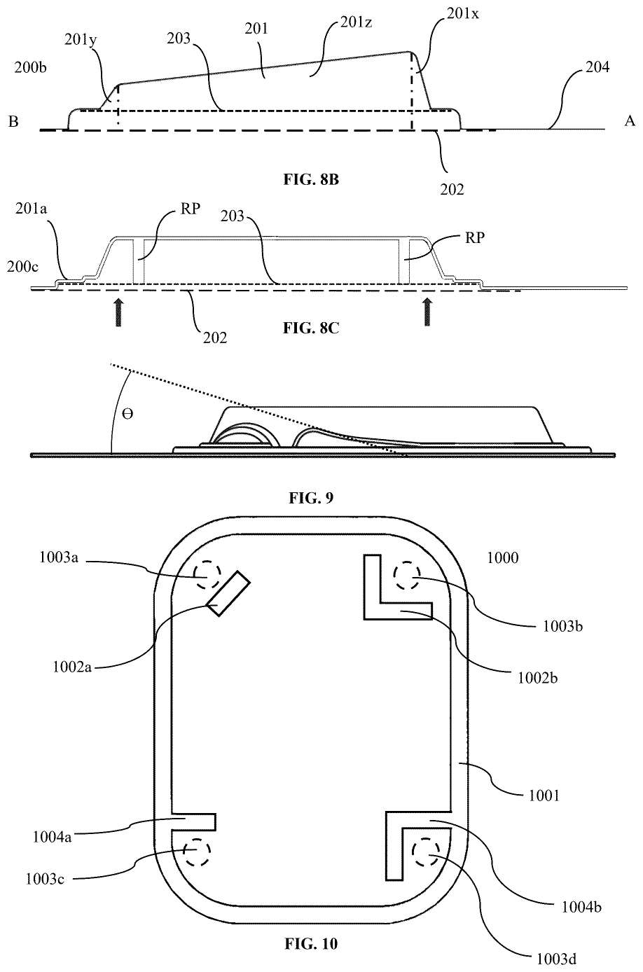

FIG. 8A shows a cross-section of an alternative volatile composition cartridge 200a, in which the sealing substrate 203a is sealed to an exterior of the cartridge, such that it encloses the membrane 202. The scaling substrate 203a may be ruptured before use, for example by a rupturing protrusion as described herein. The sealing substrate 203a may also be removed before use. Rupture or removal of the sealing substrate 203a exposes the membrane 202 and allows the volatile composition to volatilise to a surrounding environment.

FIG. 8B shows a cross-section of another alternative volatile composition cartridge 200b, which broadly corresponds to the cartridge 200 of FIG. 7 except in that the reservoir 201 has a different shape. The reservoir 201 comprises a gripping tab-end portion 201x, an opposing-end portion 201y, and a middle portion 201z therebetween. Each of the gripping tab-end portion 201x, the opposing-end portion 201y, and the middle portion 2012, have a depth perpendicular to the membrane, the depth of the middle portion 201z decreasing with increasing distance from the gripping tab. As a reservoir having this shape interacts with a corresponding housing, the shape of the reservoir will force the cartridge away from the part with which the reservoir is interacting. This may serve to force the cartridge into a rupturing protrusion, in the same manner as for a sloped section described above.

FIG. 8C shows a cross-section of another alternative volatile composition cartridge 200c, which corresponds to that of FIG. 7 except that it comprises reservoir protrusions RP extending from the reservoir to the sealing substrate 203. When a force is applied through the membrane 202 in the direction of the arrows (e.g. by a rupturing protrusion of a housing), the reservoir protrusions RP will support the sealing substrate 203 (which is also sealed to the shoulder region 201a), facilitating rupture of the sealing substrate 203. The reservoir protrusions RP may have any appropriate shape and may extend from any appropriate region of the reservoir, for example corresponding to the peninsula and island areas in FIG. 10 described herein.

FIG. 9 is an enlarged version of View II of FIG. 7, showing the maximum angle θ defined by the sloped section relative to the membrane.

FIG. 10 shows four possible configurations of a sealing substrate 1000, having a scaling area in which a peripheral region 1001 of the sealing substrate is sealed to a reservoir (not shown) to form an outer sealing area corresponding to peripheral region 1001. FIG. 10 shows a first side of the sealing substrate 1000, where the reverse side may be sealed to a reservoir as depicted in FIG. 7. In the top left corner, an island area 1002a is shown, in which the sealing substrate 1000 is sealed to the reservoir. The island area 1002a increases the tautness of the sealing substrate 1000 between the island area 1002a and the outer sealing area 1001, thereby creating a first rupturing area 1003a. The top right corner shows an alternative island area 1002b, creating a second rupturing area 1003b. In the bottom left corner, a first peninsula area 1004a is shown, corresponding generally to that created by peninsula part 201c of FIG. 7, View I. The first peninsula area 1004a creates a third rupturing area 1003c. While the first peninsula area 1004a does not surround as high a percentage of the third rupturing area 1003c as the island areas 1002a and 1002b do for the first 1003a and second 1003b rupturing areas, the first peninsula area 1004a still increases the tautness of the sealing substrate by a sufficient degree to create the third rupturing area 1003c. The bottom right corner shows a second peninsula area 1004b, which together with the outer scaling area, encloses a high proportion of the sealing substrate, thereby creating a fourth rupturing area 1003d having greater tautness than the third rupturing area 1003c, meaning that the fourth rupturing area 1003d is easier to rupture. A person skilled in the art will understand that FIG. 10, shows mere examples of possible peninsula and island areas, and other shapes and configurations may be used in the invention. For example, the second peninsula area 1003d may be extended to totally surround the fourth rupturing area 1003d, thereby creating an “island” of sealing substrate that is held taut. For the avoidance of doubt, the dashed circles representing rupturing areas 1003a, 1003b, 1003c and 1003d are indicative, and do not correspond exactly to a shape of an area that would be ruptured.

FIG. 11 shows a first sealing substrate 1101 and a second sealing substrate 1102, each in both of a vertical and horizonal alignment. Each of the first and second sealing substrates have rupturing areas depicted by dashed circles that may be generated by one or more peninsula or island areas (not shown) as described above. The first sealing substrate 1101 has rupturing areas at two opposing corners, while the second sealing substrate 1002 has rupturing areas aligned along a centre axis. When adhering products to a surface, consumers are known to prefer either vertical or horizontal alignments. The first sealing substrate 1001 has a rupturing area near the bottom of the scaling substrate in each of the vertical and horizontal alignment. Thus, when the rupturing areas of the first sealing substrate 1101 are ruptured, substantially all of a liquid volatile composition will be able to pass through the ruptured holes and contact a membrane of a volatile composition cartridge in both a vertical and horizontal alignment. As a result, the liquid volatile composition will be able to drain down to the dashed lines L1 (vertical) and L2 (horizontal), and a low amount of liquid will remain unable to pass through the sealing substrate. While the second scaling substrate 1102 can fully drain down to line L3 when aligned in a vertical configuration, it cannot fully drain when aligned in a horizontal configuration, because the rupturing areas are located half-way up the scaling substrate in this alignment, such that liquid volatile composition can only drain to the dashed line L4.

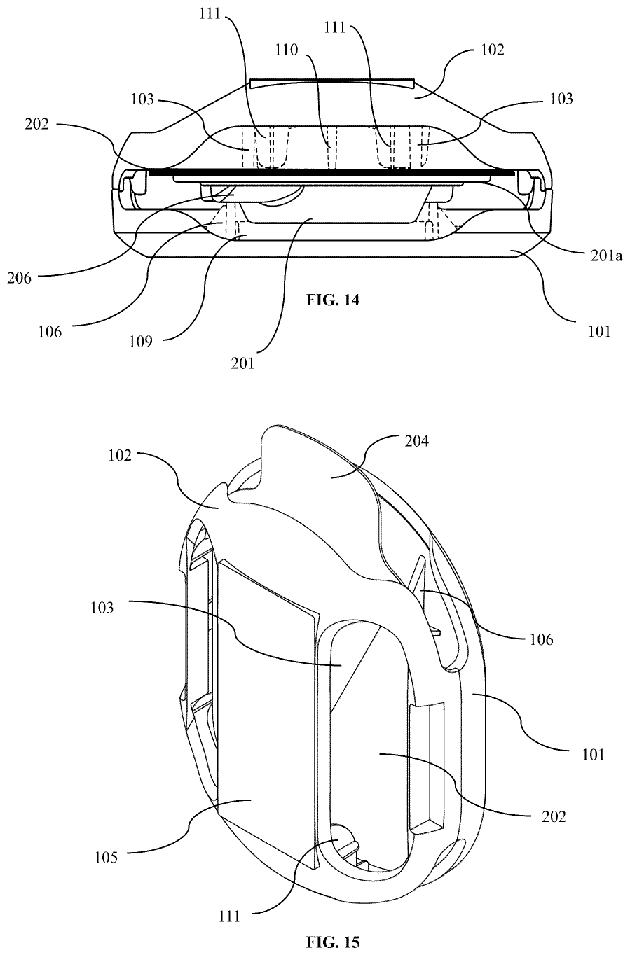

FIGS. 12 to 15 show a volatile composition dispenser 300, formed by inserting the cartridge 200 of FIG. 7 into the housing 100 of FIGS. 1 to 4. FIG. 14 depicts the internal components of the housing in thin dashed lines, to aid in distinguishing the components of the housing 100 from the cartridge 200, and the membrane is depicted with a thick solid line 202. The rupturing protrusions 103 contact the membrane 202 of the cartridge 200, deforming the membrane and rupturing the sealing substrate 203. The rupturing protrusions 103 deform the membrane 202 and extend into the space defined by the raised areas 206, thereby rupturing the sealing substrate. The membrane 202 rests in contact with the rupturing protrusions 103, and thus is distanced from the airflow openings 104, allowing an effective airflow within the housing and over the membrane 202. The gripping tab 204 extends out of the opening O, allowing it to be easily gripped by a user seeking to remove the cartridge from the housing. The reservoir 201 is received by, and visible through, the window 108. The blocking protrusion 110 can be clearly seen in FIG. 14, and the blocking protrusion 110 prevents insertion of the cartridge 200 in the opposite configuration, since in that case the reservoir 201 would be blocked by the blocking protrusion 110.

FIG. 16 shows the relationship between a sagitta, chord and arc length.

FIG. 17 shows a volatile composition dispenser adhered to a surface, and depicts a user using a pinch grip to hold the gripping tab. FIG. 17 demonstrates the improved ergonomics provided by ensuring the presence of a space between the gripping tab and the back portion of the housing. As shown in the left image, this space provides room for a user's index finger to reach behind the gripping tab. In contrast, the right image shows a housing in which the back portion is very close to the gripping tab, and there is insufficient room for a user's forefinger to properly hold the gripping tab. As a result, the finger collides with the back portion of the housing. FIG. 17 also demonstrates the advantageous ergonomics provided by the space between the gripping tab and the front portion of the housing, which allows room for a user's thumb to approach the gripping tab.

Certain benefits and advantages of the invention are illustrated by the below Examples, which are not to be construed as limitative.

### EXAMPLES

### General Materials and Methods

The Examples below were conducted using Perfume A, which is a mixture comprising: 41% esters, 33% alcohols and 20% carbonyls, with the balance being composed of various minor components. Of the 20% carbonyls, 14% is composed of volatile aldehydes and ketones having a vapor pressure of at least 0.02 Torr at 25° C., with the remaining 6% being composed of other carbonyls. These volatile aldehydes and ketones are able to react with malodor-causing amines and thiols to reduce malodor.

The components of Perfume A have the following distribution of carbon chain lengths:
19% carbon chain length of from 6 to 8;
62% carbon chain length of from 9 to 11; and
15% carbon chain length of from 12 to 14,
   with the balance being composed of small amounts of other chain lengths.

### Calculation of Perfume Weight Loss

The following apparatus was used during calculation of the perfume weight loss values detailed in Table 1:
1. Balance (Scale: Ohaus AA210 S/N 11131122540) or equivalent.
2. Housing as described hereinbelow.
3. Volatile composition cartridges as described hereinbelow, containing 4.25 ml of perfume A.
4.3M Scotch Weld Applicator TC and glue, #3797-TC or equivalent.
5. 3M Double-sided adhesive or equivalent.
6. Ikea KNÖCKLA 50 L waste bin or equivalent.
7. Room to accommodate waste bin with the following measurements, air flow, temperature/relative humidity or equivalent:
a) Laboratory Dimensions: 32 feet 4 inches long×72 inches wide×108 inches high or 1,730 ft³
b) Air Flow (Intake and Exhaust)
   Normal Mode: Average Intake Supply: 103.75 ft³/min+6%
   Average Exhaust: 149.25 ft³/min+6%
   Difference results in negative air pressure: −45.5
   Negative pressure indicates that air supply to laboratory and from an adjacent hallway or room is exhausted through the ventilation system.
c) Temperature and % Relative Humidity
   Average Temperature: 23° C.±0.1° C.
   Average % Relative Humidity: 45%±0.5%

The procedure to determine weight loss is as follows:
1. Load a cartridge with the volatile composition in such a way as to provide a sealed cartridge where the membrane is not yet wetted. For instance, one may pierce a volatile composition cartridge by cutting in it a hole that allows for insertion of an 18 gauge needle.
2. Fill the cartridge with 4.25 ml of Perfume A. This is equivalent to 4038 mg of Perfume A, which was used as the standard perfume for all experiments described herein. The volume may need to be adjusted based on the density of the composition of interest.
3. Seal the insertion hole with hot melt adhesive.
4. Measure and record the weight of the apparatus.
5. Insert the cartridge into a housing for holding and orienting the cartridge, and ensure that the cartridge is set correctly within the housing to ensure proper air flow therethrough.
6. Activate the cartridge by any appropriate means to allow contact between the perfume and the membrane, thereby wetting the membrane.
7. Stick 1 side of the adhesive on the housing and the other side on the waste bin lid.
8. Open the waste bin for 10 seconds for 25 separate times every day at regular intervals.
9. Record the cartridge weight daily at the same time for a specific time period.
10. Determine the weight loss of the volatile composition during the relevant time period.

### Calculation of Perfume Concentration in Waste Bin

The perfume concentration (ppm) in waste bin is calculated based on following:

$$1. Perfume concentration ( ppm ) in waste bin = Moles of perfume in \\ \\ waste bin / Moles of air in waste bin * 1 , 000 , 000$$

$$2. Moles of perfume in waste bin = Amount of perfume in waste bin / Molecular weight of perfume$$

$$3. Moles of air in waste bin = Volume of waste bin / Molar volume of \\ \\ air at room temperature$$

$$4. Amount of perfume in waste bin = Mass of perfume evaporated per \\ \\ hour / effective air change per hour$$

$$5. Mass of perfume evaporated per hour = Mass of perfume evaporated over test period ( grams ) / number of hours during test period$$

6. In this work, the following values were used:

$$a . Molecular weight of perfume = 150 g / mol$$

$$b . Volume of waste bin = 50 L$$

$$c . Molar volume of air at room temperature = 24.5 L / mol$$

$$d . Effective air exchange per hour = 1.1$$

$$e . Number of hours during test period = 672 hours ( 28 days )$$

### Waste Bin Adhesive Test Method

The following apparatus was used during testing to determine if adhesive has sufficient adhesive strength for use in a waste bin:
1. Housing as described hereinbelow.
2. Volatile composition cartridges as described hereinbelow, containing 5 ml of perfume A.
3. Desired adhesive.
4. Ikea® KNÖCKLA 50 L waste bin (polypropylene) or equivalent.
5. Oki Pan-ola™ Canola Oil Cooking Spray or equivalent.
6. Room to accommodate waste bin with the following measurements, temperature/relative humidity or equivalent:
a) Laboratory Dimensions: 32 feet 4 inches long×72 inches wide×108 inches high or 1,730 ft³
b) Temperature and % Relative Humidity
   Average Temperature: 35° C.±0.1° C.
   Average % Relative Humidity: 60%±0.5%

The procedure to determine if adhesive has sufficient adhesive strength for use in a waste bin is as described:
1. Insert the cartridge into a housing for holding and orienting the cartridge, and ensure that the cartridge is set correctly within the housing to ensure proper air flow therethrough.
2. Activate the cartridge by any appropriate means to allow contact between the perfume and the membrane, thereby wetting the membrane.
3. Gently spray a layer of cooking spray on the lid of the waste bin, then use a paper towel to spread the cooking spray evenly across the lid.
4. Remove excess cooking spray on the lid using another paper towel.
5. Stick 1 side of the adhesive on the housing, then stick the other side of the adhesive on the waste bin lid.
6. Close the waste bin lid and place the waste bin inside the 35° C. laboratory for 24 hours.
7. Open the waste bin for 10 seconds for 25 separate times every day at regular intervals for a total period of 2 months.
8. Replace the used cartridge with a new one every 7 days without changing the housing.
9. Check whether the housing stays on the waste bin lid at the end of 2 months.

### Malodor Intensity Test Method

The following apparatus was used.
1. Housing as described hereinbelow.
2. Volatile composition cartridges as described hereinbelow, containing 5 ml of perfume A.
3. Ikea® KNÖCKLA 50 L waste bin (polypropylene) or equivalent
4. Malodor sources (malodor intensity evaluated within 24 hours from time of preparation):
   Dried sole fish: one 0.12 g piece on a petri dish.
   Bacon grease: 100 cm² fabric patch was infused with cooked bacon grease.
   Cooked garlic: cook 17.5 g of garlic in 0.5 cup of oil (6.25 g) and place 0.32 g of cooked garlic on petri dish.
   Broccoli: Fresh whole broccoli kept in a fridge for 5-7 days. Extract 15 g of the broccoli florets and use as-is.
   Bathroom: 20 μL of synthetic fecal and urine cocktail was placed on a petri dish.

### Test Protocol:

1. Place a waste bin liner onto waste bins, one as a control (no volatile composition dispenser) and the remaining with a volatile composition dispenser.
2. For the waste bins with a volatile composition dispenser, adhere the housing to the centre of the lid of the waste bin at least 16 hours but not more than 24 hours before evaluation.
3. Place the malodor sources into the waste bins 1 hour before evaluation and leave at room temperature.
4. For evaluation:
   a. Open the waste bin using the foot pedal and smell—this is to simulate throwing waste into bin.
   b. Panelist should take note of the smell immediately when lid is open and to evaluate smell for 5 secs.
      i. Steps to take when smelling waste bin:
         1. Panelist to step on foot pedal.
         2. Panelist should slightly bend their hips and not bend their knees when smelling the waste bin.
         3. Evaluate smell for 5 secs in that position.
         4. Immediately remove feet from foot pedal after 5 secs and ensure lid is closed.
   c. Panelists are to take a 2-minute break in between each waste bin evaluation.
   d. Evaluate waste bin with malodor only first for malodor intensity and evaluate and identify types of malodor present.
   e. Then evaluate waste bin with malodor and volatile composition dispenser for perfume intensity and malodor intensity.

The malodor intensity was scored as follows.
0: No malodor present
10: I think there is malodor present (unsure)
20: I detect something, but can I recognize it?
25: Slight malodor present
40: Slight to moderate malodor present
50: Moderate malodor present
60: Moderate to strong malodor present
75: Strong malodor present
100: Extremely strong malodor present

### Example 1: Volatile Composition Evaporation

In order to demonstrate the ability of a volatile composition to evaporate and counteract malodor in a waste bin, volatile composition dispensers similar to those shown in FIGS. 12-15 were prepared and affixed to the interior wall of a waste bin. The volatile composition dispensers differed from FIGS. 12-15 in that the shape of the protrusions was different, but the exterior shape of the housing, reservoir, membrane, and parameters A_{E}, A_{F}, and D2 as described herein were equivalent to those shown in FIGS. 12-15. The volatile composition dispensers had differing membrane evaporative surface areas, total areas of the airflow openings, and distances between the membrane and the wall of the waste bin. The volatile composition dispensers were left inside the waste bin for a period of four weeks, and the weight loss and perfume concentration in the waste bin were determined according to the Calculation of Perfume Weight Loss and Calculation of Perfume Concentration in Waste Bin methods described above. Results are shown in Table 1 below.

Four of the tested dispensers were assessed for malodor intensity according to the Malodor Intensity Test Method above. All of the four tested dispensers provided reduced malodor intensity compared to the control.

TABLE 1

| | | Mean | | | | |
|---|---|---|---|---|---|---|
| | | distance | | | | |
| Total | Membrane | from waste | | | 4 week | Malodor |
| airflow | evaporative | bin wall to | D2 × A_{E} × A_{F} | Weight | Average | Intensity |
| opening | surface | membrane, | (calculated | Loss | Perfume | Judged by |
| area, “A_{F}” | area, “A_{E}” | “D2” | from | after 4 | Concentration | Expert |
| (rounded to | (rounded to | (rounded to | unrounded | weeks | in waste bin | Panelist |
| nearest cm²) | nearest cm²) | nearest mm) | values) | (g) | (ppm) | (control: 60) |
| 7 | 10 | 5 | 352 | 0.550 | 2.4 | |
| 7 | 10 | 10 | 704 | 0.670 | 3.0 | |
| 7 | 10 | 12 | 845 | 0.746 | 3.3 | |
| 7 | 10 | 15 | 1056 | 0.895 | 4.0 | 45 |
| 10 | 10 | 5 | 520 | 0.812 | 3.6 | |
| 10 | 10 | 10 | 1039 | 0.988 | 4.4 | 40 |
| 10 | 10 | 12 | 1247 | 1.101 | 4.9 | |
| 10 | 10 | 15 | 1559 | 1.568 | 6.9 | |
| 15 | 27 | 5 | 2053 | 1.421 | 6.3 | 20 |
| 15 | 27 | 10 | 4107 | 1.730 | 7.6 | |
| 15 | 27 | 12 | 4928 | 1.928 | 8.5 | 0 |
| 15 | 27 | 15 | 6160 | 2.127 | 9.4 | |

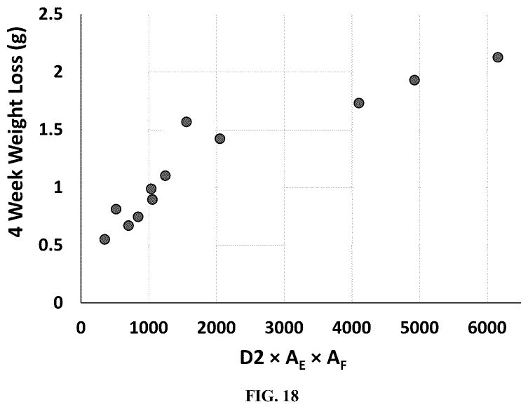

Correlation of the value D2×AB×Ar against weight loss over four weeks is shown in FIG. 18.

The results in Table 1 demonstrate that the ability of the tested volatile composition dispensers to volatilise a volatile composition, and hence counteract malodor, improves with the increased value of D2×A_{E}×A_{F}. In particular, volatile composition dispensers for which D2×A_{E}×A_{F} is above 1100 (e.g. above 1500, and especially above 2000) had excellent evaporative properties and provided sufficient perfume in the waste bin to effectively counteract malodor, with volatile composition dispensers for which D2×A_{E}×A_{F} is above 4500 providing the best counteracting of malodor.

### Example 2: Adhesive Properties

Volatile composition dispensers as used in Example 1, were prepared, and completed with a 12 cm³ strip of adhesive on the adhering section.

There are two possible adhesive failure modes: the adhesion between the adhesive and the housing, and the adhesion between the adhesive and the surface to which it is attached, typically the interior of a waste bin.

The adhesive strength as measured by ASTM D3330-04(2018) for different adhesives is provided in Table 2 below, along with results for the Waste Bin Adhesive Test Method described above. Also shown are associated physical properties of the adhesive that affect the adhesive strength, including Total Surface Energy as measured by ASTM D7490-13(2022), and Polar Ratio, which is the ratio of polar SE over total SE. Strong adhesion can be achieved by the adhesive having both (i) a total surface energy lower than that of the surface that it adheres to; and (ii) matching surface energy type between the adhesive and the surface.

TABLE 2

| | | 90° Peel | | |
|---|---|---|---|---|
| | Waste Bin | Adhesion | Associated physical properties | |
| | Adhesive | (N/cm) | Total Surface | |
| | Test Result | [ASTM | Energy (mN/m) | Polar Ratio |
| | (Polypropylene | D3330- | [ASTM D7490- | (polar SE/ |
| Adhesive | Surface) | 04(2018)] | 13(2022)] | total SE) |
| 3M ® 9495LE | Failed | 10 | 17.12 | 5.61% |
| 3M ® VHB F9473-PC | Failed | 16 | 21.82 | 17.60% |
| 3M ® VHB 4910 | Failed | 26 | 32.72 | 4.16% |
| 3M ® VHB 4941 | Passed | 39 | 19.74 | 0.05% |
| 3M ® VHB 4950 | Passed | 44 | 18.06 | 0.11% |
| 3M ® VHB 4945 | Passed | 44 | 20.05 | 0.30% |
| 3M ® VHB LSE-160WF | Passed | 54 | 19.85 | 2.42% |

As can be seen from the results in Table 2, adhesives that have a Total Surface Energy of less than 25 mN/m, and a Polar Ratio of less than 5%, provided good adhesive strength (>30 N/cm) as measured by 90° peel adhesion.

### Example 3: Surface Properties of Housing

Plastic materials such as polyethylene and polypropylene are commonly used as materials for volatile composition dispensers and waste bins. However, these types of plastic have very low total surface energy and very low polar ratio, and so it can be challenging to provide adhesives having a sufficiently low total surface energy and polar ratio in order to obtain a high adhesive strength. Therefore, different surface finishes were investigated in order to obtain plastic surfaces having a higher total surface energy. The use of a surface finish that provides an increased surface energy will more easily allow an adhesive to be chosen that has a lower total surface energy than the material in question.

Volatile composition dispensers as used in Example 1, were prepared from polypropylene, and completed with a 12 cm³ strip of 3M® VHB 4941 adhesive on the adhering section. In order to improve the adhesion of the adhesive to the housing, the adhering section may be subjected to a surface finish that increases its Total Surface Energy, and/or reduces one or both of its Polar Ratio and Surface Roughness. The effect of different surface finishes is shown in Table 3 below.

TABLE 3

| | Force Needed | Associated physical properties | | |
|---|---|---|---|---|
| | to Remove | Total Surface | | Surface |
| | Adhesive | Energy (mN/m) | Polar Ratio | Roughness |
| Polypropylene | from Housing | [ASTM D7490- | (polar SE/ | (μm) [ISO |
| Surface Finish | (N) | 13(2022)] | total SE) | 21920-1: 2021] |
| VDI27 | 37 | 30.44 | 5.19% | 1.6443 |
| Glossy | 55 | 32.08 | 0% | 0.2035 |
| Plasma-treated | 69 | 43.92 | 4.62% | NT |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular configurations of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A housing for a volatile composition dispenser, the housing comprising:
a front portion and a back portion, where the back portion comprises at least one airflow opening, and the front and back portions together define an interior space for receiving a volatile composition cartridge;
one or more rupturing protrusions extending from an interior surface of the back portion towards the interior space, the one or more rupturing protrusions for directly rupturing a sealing substrate of a volatile composition cartridge;
one or more supporting protrusions extending from an interior surface of the front portion towards the interior space, the one or more supporting protrusions for supporting a volatile composition cartridge in order to facilitate rupturing of the sealing substrate by the one or more rupturing protrusions; and
a cartridge insertion opening that allows a volatile composition cartridge to be slidably received by the interior space.

2. The housing according to claim 1, wherein the one or more rupturing protrusions comprise a rupturing apex for rupturing a sealing substrate of a volatile composition cartridge.

3. The housing according to claim 2, wherein the rupturing apex comprises an arc having an arc radius of less than 0.9 cm,
optionally wherein the rupturing apex comprises an arc having an arc radius of less than 0.7 cm,
more optionally wherein the rupturing apex comprises an arc having an arc radius of less than 0.5 cm.

4. The housing according to claim 2, wherein the rupturing apex comprises an arc, and where a chord defined by a sagitta of 1.025 mm has an arc length of from 4 mm to 8.5 mm.

5. The housing according to claim 2, wherein the one or more rupturing protrusions comprise a sloped region facing the cartridge insertion opening, where the sloped region is configured such that as a cartridge is inserted into the interior space via the cartridge insertion opening, the sloped region guides the cartridge towards the rupturing apex of the one or more rupturing protrusions.

6. The housing according to claim 1, wherein the one or more rupturing protrusions comprises at least two rupturing protrusions.

7. The housing according to claim 6, wherein the at least two rupturing protrusions are laterally offset from a line bisecting the housing in a longitudinal direction.

8. The housing according to claim 7, wherein the at least two rupturing protrusions are laterally and longitudinally offset from each other.

9. The housing according to claim 1, wherein the one or more supporting protrusions comprise a sloped region facing the cartridge insertion opening, where the sloped region is configured such that as a cartridge is inserted into the interior space via the cartridge insertion opening, the sloped region directs the cartridge towards the one or more rupturing protrusions.

10. The housing according to claim 1, wherein at least one of the one or more rupturing protrusions is closer to the cartridge insertion opening than one of the one or more supporting protrusions.

11. The housing according to claim 1, wherein at least one of (a) and (b) applies:
(a) a cartridge contacting area of the one or more rupturing protrusions is smaller than a cartridge contacting area of the one or more supporting protrusions; and
(b) the one or more rupturing protrusions and the one or more supporting protrusions each have an arc defined by a chord at a sagitta of 1.025 mm, and a corresponding arc length of the one or more rupturing protrusions is shorter than a corresponding arc length of the one or more supporting protrusions.

12. The housing according to claim 1, wherein each of the one or more rupturing protrusions and one or more supporting protrusions are arranged in pairs, such that each rupturing protrusion has a corresponding supporting protrusion.

13. The housing according to claim 12, wherein a distal point of each rupturing protrusion is less than 1.5 cm from a distal point of its corresponding supporting protrusion,
optionally less than 1 cm from its corresponding supporting protrusion,
more optionally less than 0.7 cm from its corresponding supporting protrusion.

14. The housing according to claim 1, further comprising a blocking protrusion extending from an interior surface of the front or back portions, the blocking protrusion for preventing insertion of a volatile composition cartridge in an undesired orientation.

15. The housing according to claim 1, wherein the front portion comprises a window,
optionally wherein the window is at least partially bordered by one or more guiding protrusions, the guiding protrusions for aligning a volatile composition cartridge as the volatile composition cartridge is inserted into the housing.

16. The housing according to claim 1, wherein:
the one or more rupturing protrusions each comprise a rupturing apex for rupturing a sealing substrate of a volatile composition cartridge;
the one or more rupturing protrusions comprise a sloped region facing the cartridge insertion opening, where the sloped region is configured such that as a cartridge is inserted into the interior space via the cartridge insertion opening, the sloped region guides the cartridge towards the rupturing apex of the one or more rupturing protrusions;
the one or more supporting protrusions comprise a sloped region facing the cartridge insertion opening, where the sloped region is configured such that as a cartridge is inserted into the interior space via the cartridge insertion opening, the sloped region guides an opposing side of the cartridge towards the rupturing apex of the one or more rupturing protrusions; and
at least one of the one or more rupturing protrusions is closer to the cartridge insertion opening than any of the one or more supporting protrusions.

17. The housing according to claim 16, wherein each of the one or more rupturing protrusions and one or more supporting protrusions are arranged in pairs, such that each rupturing protrusion has a corresponding supporting protrusion.

18. The housing according to claim 1, wherein the housing does not comprise moving parts.

19. A volatile composition dispenser comprising:
a housing as defined in any one of the previous claims; and
a volatile composition cartridge, the volatile composition cartridge comprising:
a reservoir containing a volatile composition;
a sealing substrate enclosing the reservoir; and
a membrane enclosing the reservoir, the membrane configured to allow volatilisation of the volatile composition,
wherein:
the volatile composition dispenser is configured such that as the volatile composition cartridge is slid into the housing via the cartridge insertion opening, the at least one rupturing protrusion is configured to rupture the sealing substrate without rupturing the membrane.

20. The volatile composition dispenser according to claim 19, wherein the volatile composition cartridge comprises a sloped section on a side of the volatile composition cartridge that is opposing to the membrane,
wherein the sloped section is for engaging with the one or more supporting protrusions, such that as the volatile composition cartridge is slid into the housing via the cartridge insertion opening, the sloped section engages with one of the one or more supporting protrusions and directs the volatile composition cartridge in a direction towards the one or more rupturing protrusions, thereby increasing the force exerted by the rupturing protrusion on the sealing substrate as compared to a force exerted when the sloped section is not engaged with one of the one or more supporting protrusions.
